(19) United States
(12) Patent Application Publication
(10) Pub. No.: US 2025/0248737 A1
(43) Pub. Date: Aug. 7, 2025

(54) FLEXIBLE ACCESS SUPPLEMENTATION TUBES FOR ENDOSCOPIC EXPOSURE AND RETRACTION

(71) Applicant: Amazing Brace LLC, Tampa, FL (US)

(72) Inventor: Victor Hayes, Tampa, FL (US)

(21) Appl. No.: 19/046,643

(22) Filed: Feb. 6, 2025

Related U.S. Application Data
(60) Provisional application No. 63/550,552, filed on Feb. 6, 2024.

(51) Int. Cl. A61B 17/34; A61B 1/018; A61B 1/05; A61B 1/313; A61B 17/00; A61B 17/02; A61M 25/00; A61M 25/01

(52) U.S. Cl. CPC A61B 17/3423; A61B 1/018; A61B 1/05; A61B 1/3132; A61B 2017/00862; A61B 2017/0225; A61M 2025/0024; A61M 25/005; A61M 2025/0177

(57) ABSTRACT

Flexible access supplementation tubes for endoscopic exposure and retraction that include at least a first flexible tube comprising a material suitable for insertion into a human, and a second flexible tube comprising a material suitable for insertion into a human, in which the first and second flexible tubes are interconnected with each other, and in which at least one of the first flexible tube or the second flexible tube is a size suitable to accommodate an instrument therein. Also provided are kits that include the present devices, and methods of using the present devices on a patient in need thereof.

### FIELD

The present application relates to new flexible access supplementation tubes for endoscopic exposure and retraction (FASTER), kits that include such devices, and methods of using the same by treating patients by utilizing such tubes.

### BACKGROUND

Most endoscopic spinal surgery sets have a scope with a large working outer diameter and instruments must be passed through these rigid scopes. Arthroscopic procedures typically use a fixed, rigid arthroscope with multiple working portals and/or cannulated endoscopes with working channels. These working channels are very small, which limits the type, size and trajectory of instruments that can be used in orthopedics. This issue is particularly magnified in endoscopic spinal surgery. Because the spinal openings are so small, working with the rigid scope makes it difficult to access many areas of the spine. It also makes it difficult to pass instruments through the smaller rigid working channels. However, as endoscopes have become smaller, the need for different access in minimally invasive surgery has become paramount.

### SUMMARY

Provided herein are Flexible Access Supplementation Tubes for Endoscopic (Exposure and) Retraction (also referred to herein as “FASTER”) devices and systems. Also provided are kits that include one or more of the present devices, and methods of using the present devices by treating patients by utilizing such devices and systems. According to non-limiting example embodiments, the present methods are methods of performing endoscopic or open surgery on the spine or extremities of a patient while utilizing the present FASTER devices and systems.

The present FASTER devices and systems include at least a first flexible tube made of a material suitable for insertion into a human, and a second flexible tube made of a material suitable for insertion into a human, in which the first and second flexible tubes are interconnected with each other, and in which at least one of the first flexible tube or the second flexible tube is a size suitable to accommodate an instrument therein. The present devices and systems may include a third flexible tube interconnected with the first and second flexible tubes.

Also provided are kits that include at least one flexible access supplementation tube for endoscopic exposure and retraction according to embodiments herein, and at least one additional component selected from the group consisting of instructions for use of the at least one flexible access supplementation tube for endoscopic exposure and retraction, a storage container, a guidewire, a visualization tool, and a working instrument.

Also provided are methods of treating a patient that include inserting a device into a patient, in which the device includes at least a first flexible tube and a second flexible tube, in which the first and second flexible tubes are interconnected with each other; and inserting a first instrument into the first flexible tube, to deliver the device to a predetermined location to a patient in need thereof. The tubes will be expandible to varing degrees based by using Nitinol sleeves that can be introduced inside the tubular component, then deployed and expanded to varing degrees based on the diameter of exposure needed. The nitinol expander may be fully or partially deployed to minimize tissue retaction while keeping the external incisions small.

Nitinol stents are commonly used in medicine, but these devices are typically expanded within a vessel that is narrowed to widen the lumen. The present devices are novel at least because they use expandible nitinol tubes deployed within a sleeve for use in working channel retractor systems.

### BRIEF DESCRIPTION OF THE FIGURES

FIG. 1 depicts a cross-sectional view of a rigid cannula configured for inserting a camcra/inflow/light into a patient, and a cross-sectional view of an embodiment of the present systems/devices for delivering camera/inflow/light into a patient, which does not include an outer cannula.

FIG. 2 depicts an embodiment in which one sleeve is over a traditional endoscope.

FIG. 3 depicts examples of flexible materials suitable for insertion into a human body, including Braided Composite Tubes (BCT) with Nitinol, Nitinol alone, and elastic polymer tubes that may be reinforced with metal.

FIG. 4 depicts various configurations of tubes according to example embodiments, which may be expandable and fixed depending on needs during surgery.

FIG. 5 depicts embodiments in which braided composite systems/devices may include Nitinol tubes inside a braided outer tube, braided composite systems combined with an elastomer system, or multiple elastomer systems.

FIGS. 6-10 depict multiple examples of elastic polymer tubes in accordance with the present application, with an internal seal and accessory IV tube.

FIGS. 11-12 depict examples of a bi-portal elastic polymer system, from a side perspective view and from a cross section view.

FIGS. 13-15 show examples of tri-tube embodiments of the present application. FIG. 16 depicts an example of an elastic polymer tube.

FIG. 17 depicts an example bi-portal interconnected FASTER system in which the tubes are made of multiple materials, depending on their use.

FIG. 18 depicts an example instrument in which a camera portal can be pointed at working “jaws”, which may be used in the present systems/devices.

FIGS. 19-20 depict embodiments, which include interconnected tubes, in which at least one of the tubes is sized and configured to slide onto an instrument, and at least one additional interconnected tube is available as an accessory channel.

FIG. 21 shows embodiments in which one of the sheaths/tubes of the present system/device slides over a first instrument, and then another (or multiple) interconnected sheath/tube/accessory channel is available for a second instrument to be inserted so the second instrument is interconnected to the first.

FIG. 22 depicts embodiments including a Nitinol tube and shows how an additional removable catheter sheath may be included in the original device to maintain a Nitinol tube in a closed position until it is desired to deploy the Nitinol tube into an expanded position.

FIG. 23 depicts potential modifications of embodiments of the present application, which may include for example an expandible Nitinol material.

FIGS. 24-25 show a cross-sectional view of embodiments using Nitinol as a working channel.

FIGS. 26-28 show examples of the present systems/devices in use, in which an expandable Nitinol tube is used.

FIG. 29 depicts examples of the present FASTER devices and systems.

FIGS. 30-32 depict examples of a Kerrison with a present FASTER system/device attached (and not attached—see FIG. 31).

FIG. 33 depicts examples of passing a scope within IV tubing and an isolated tube and shows an example of flexibility.

FIG. 34 shows an example of a scope and secondary fluid channel incorporated into a tube in accordance with examples.

FIG. 35 shows a plastic round holder that can be used to turn and spin the interconnected tubes of the present systems and devices.

FIG. 36 shows that the present systems/devices may be relatively small in size.

FIGS. 37A and 37B depict example embodiments of the present FASTER system (FIG. 37B on the right) vs. a traditional rigid scope on the left (FIG. 37A).

FIGS. 38 and 39 compare the use of an endoscope in a standard tube (FIG. 38) vs. in a flexible tube (FIG. 39), which allows for more flexibility.

FIG. 40 shows an embodiment of a FASTER device/system of the present application.

FIG. 41 shows another example embodiment of the present system, in a tri-tube form.

FIG. 42 shows an embodiment in which a tube of the present FASTER device/system slides onto an instrument and an interconnected tube is available for insertion of a e.g., a scope.

FIG. 43 depicts how an example bi-portal dual cannula device of the present application can be used when working in the neuroforamen for a transforaminal approach.

FIG. 44 depicts an embodiment having an adjustable flexible fingertip, which may be used with embodiments of the present systems and devices, e.g. to function to bend the tip of the interconnected tubes to help navigate the spinal canal.

FIGS. 45-48 relate to additional FASTER instruments that may be used in accordance with the present application. In particular, these figures show Standard Insertable Self Drilling Cannula on T-handle for Spine Instrument passage.

FIG. 45 depicts an embodiment having a T-handle that can disconnect from a clip in metal solid stylet with a cutting flute (Self Drilling).

FIG. 46 shows an embodiment having a working channel cannula that meets with a T-handle to become one solid device that can be used to cannulate the facets and/or pedicle according to example embodiments.

FIGS. 47-48 shows an assembled instrument according to example embodiments, locked in and ready for insertion through bone, transpedicular and/or transforaminal to disc.

FIG. 49 is a table showing example outer and inner diameter of cannulas for the different cages.

FIG. 50 shows an example of a self-drilling cannula on T-handle drilling through sawbones and showing the trajectory of approach for radiographic guided transpedicular approach to remove disc pathology and/or bone spurs.

FIG. 51 shows an embodiment having of a cannula locked in bone to allow working instruments and/or scope to be passed from skin down to the surgical site and posterior disc.

FIG. 52 shows example combined transpedicular and transforaminal approaches to access posterior spinal structures.

FIG. 53 shows a standard YESS (Yeung Endoscopic Spine Surgery System) endoscopic fixed working cannula arthroscope and its limitations compared to the novel FASTER features.

### DETAILED DESCRIPTION

The terms “a” or “an”, as used herein, are defined as one or more than one. The term another, as used herein, is defined as at least a second or more. The terms “including” and “having,” as used herein, are defined as comprising (i.c., open language).

In addition to being described herein, various embodiments are depicted in the figures.

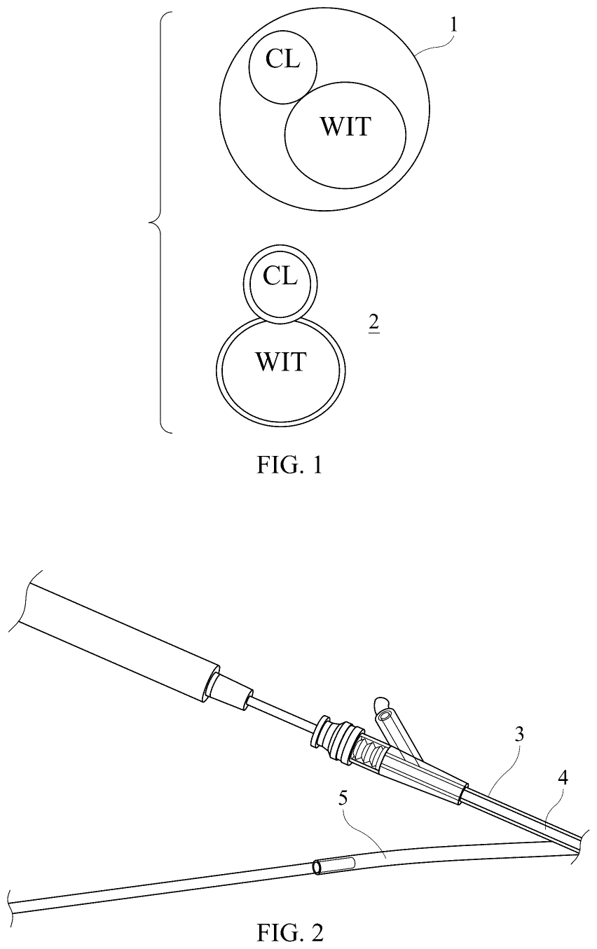

A series of instruments is required to perform an endoscopic spine surgery (and other minimal invasive surgeries) and most spine endoscopes have working channels for the passage of various instruments. This configuration increases the OD (outer diameter) of a device and limits the size of instruments that can be used in a procedure. See e.g., the top of FIG. 1, which shows a rigid cannula 1 having both a camera/inflow/light (“CIT”) and a working instrument tub (“WIT”) within the same rigid cannula.

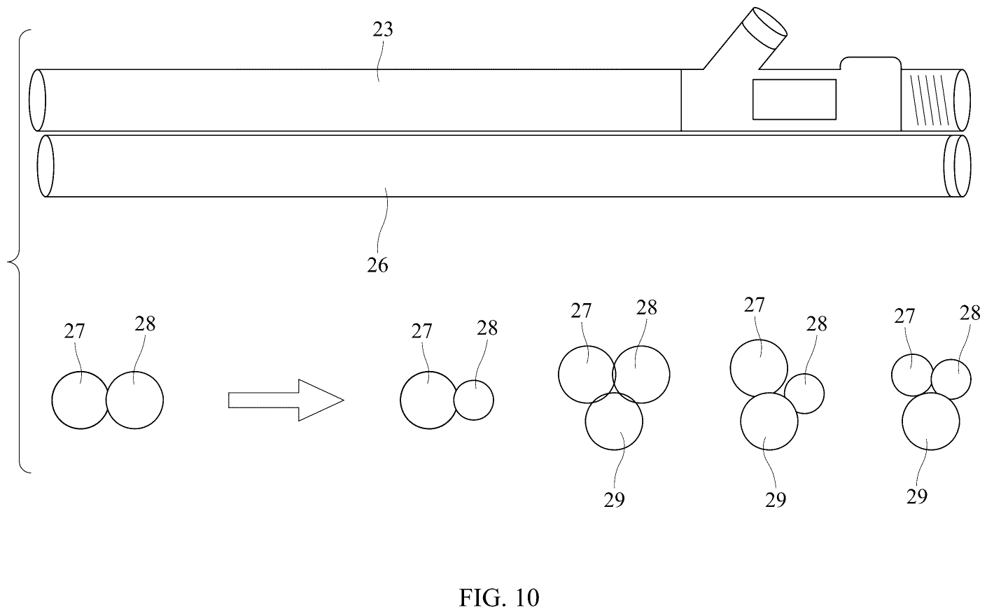

In example embodiments, using clastic polymer tubes (with expandible options), or other flexible tubes within the scope of the present application, one can traverse a body, such as a human body with more versatile access when compared to fixed rigid cannulas. When tubes, such as elastic polymer tubes, are interconnected with one another, the overall circumferential space (OD: Outer Diameter) required to pass the same instruments to a surgical site in a patient, can be reduced. See for example, the bottom of FIG. 1, which shows a cross section of a non-limiting example of the present system 2. In the present embodiments, surgeons can access smaller regions of the body because the tubes may have different material thicknesses, diameters, configurations (FIG. 10) and flexibilities. The present FASTER system can be attached to both open and arthroscopic instruments to enhance their utility with a simple add on modification. The present devices and systems are unique and offer a significant advantage over other products used in surgery, and in particular improve the utility of open and endoscopic instruments.

Some of the smallest arthroscopes use a “Chip on a stick” type technology, which may include imaging sensors on the distal tip of a device, such as an endoscope, for minimally invasive surgery, to change the process by which images are transmitted and reduce the OD (Outer Diameter) of the camera. “Chip on a stick” type arthroscopes have a much smaller outer diameter than the traditional scopes which require multiple lenses and a light source, but they still require additional channels to pass instruments and get the work completed with regards to surgery. Some of the smaller scopes currently on the market have an OD (outer) diameter of e.g. 1.9 mm. Inflow outflow fluid is required for visualization and this slightly increases the outer diameter of the cannula to house this arthroscope.

The following components are necessary to perform Arthroscopic/Endoscopic surgery: irrigation inflow/suction outflow, a light source, camera, and a working channel for electrocautery and/or instrument passage. Outflow suction may also be required if bleeding obstructs visualization. In current surgeries, the above additions must be incorporated inside a cannular system or multiple separate tubes, which are typically rigid. Separate incisions can be made as with extremity surgery creating a multiport technique. Another option that is used in spinal surgery, where the structures are much smaller and encased in bony structures, is to use an endoscope with a working channel built into it. This technology has not truly advanced in twenty-five years. The basics of the working channel spine scope are essentially unchanged, as it is a round/rigid tube 1 containing all important components to perform spine surgery. This working channel scope has significant limitations, the first being that it is rigid and difficult to navigate around the spine. It is also hard to reduce the scopes outer diameter in general because this one device must house all the five necessities above. Because the working scopes are rigid and have rigid working channels for instrument tunnels, too. This severely limits advancing technique in spinal surgery.

Using a working channel endoscope dramatically limits the size of the instruments used to perform a surgery. Another limitation is that tight anatomic regions cannot be traversed safely because vital structures (nerves and/or ligaments/tendons) obstruct a linear exposure “access” pathway. This makes it difficult to address different pathologies without causing iatrogenic damage to those structures.

Two very tightly constrained areas would be the spine in general and the hip joints during arthroscopy. Hip arthroscopy typically requires traction to distract or open up the joint space during arthroscopy. The spine is incased in bones and working through the natural openings (which is optimal) is difficult because of the space constraints.

In example procedures other than hip and spine, additional portals are necessary to pass working instruments, and this requires additional incisions.

When cannulas are used during surgery to introduce fluid, which is necessary for visualization and outflow, working instruments can increase the size of the outer diameter. The outer diameter of access cannulas 1 can directly affect the ability to enter small surgical sites, and this is especially true during spinal procedures. As the size of the rigid cannula 1 increases, the ability to maneuver and access smaller areas is inversely affected. A rigid working cannula 1 in spinal surgery requires a camera tunnel, inflow/outflow tunnel, and instrument tunnel (working tunnel) to perform work during surgery. By housing all of this in one or multiple rigid cannulas, the OD increases substantially. Rigid working channels also limit the ability to pass flexible instruments and to navigate freely within the surgical field.

General endoscopes or working channel spine arthroscopic scopes and/or any instruments needed for surgery can be supplemented with the present novel access systems and devices to increase the utility of the devices. With interconnected multiple hybrid portals 2 of varying flexibility and diameter, in addition to using a non-rigid material, safer access pathways are created and pathology with open and/or arthroscopic surgery, is addressed. This is novel at least because this improves on the multiple limitations of arthroscopic surgery in general, without having to create new instruments. Smaller, versatile, flexible access pathways are created with a reduced surface contact area. The surface area of the portal will have variable shapes based on the number of portals used and OD/ID (Outer diameter/Inner diameter), as shown for example, in FIG. 1. These can also be exchanged easily based on the constrictions of the space encountered.

FIG. 2 depicts a non-limiting example in which one sleeve 3 is over a traditional endoscope 4. FIG. 2 and other figures in this application happen to depict a NanoScope™ (by ARTHREX®) as the endoscope, but it should be understood that other devices may be used in accordance with the present application, and this device is depicted by way of non-limiting example. The second sleeve by portal 5, adjacent to the scope creates an additional pathway for instruments to the surgical field and avoids creating another working portal in extremity surgery. This also avoids the use of the rigid cannula to house all the working instruments.

According to non-limiting example embodiments, the present application provides novel elastic polymer tubular access devices and systems with built in inflow/outflow interconnected tubes, which may be used in orthopedic arthroscopy to avoid rigid cannula use, while allowing smaller, flexible and expandible exposure portals.

The present access tubes can be interconnected, have varied sizes, shapes and flexibility based on the materials used and the requirements of the particular surgery. They may include elastic polymer tubes (including, but not limited to IV tubes), braided Composite (Nitinol/PET strips or other braids), or stand-alone Nitinol tubes and/or used primarily as surgical access supplements to assist during surgery.

According to example embodiments of the present devices, tubes are configured such that they can be placed in tight regions and then expanded e.g., using Braided Composite Tubes (BCT) made of e.g., Woven Nitinol and (Polyethylene Terephthalate (PET) strips or Nitinol alone. This allows the tubes to be permanently enlarged so that larger instruments can be used. The tubes can be expanded to create a greater sized working channel, without removing and/or exchanging the tube for one of greater diameter during a procedure. These materials have not previously been used as working channels to perform surgery.

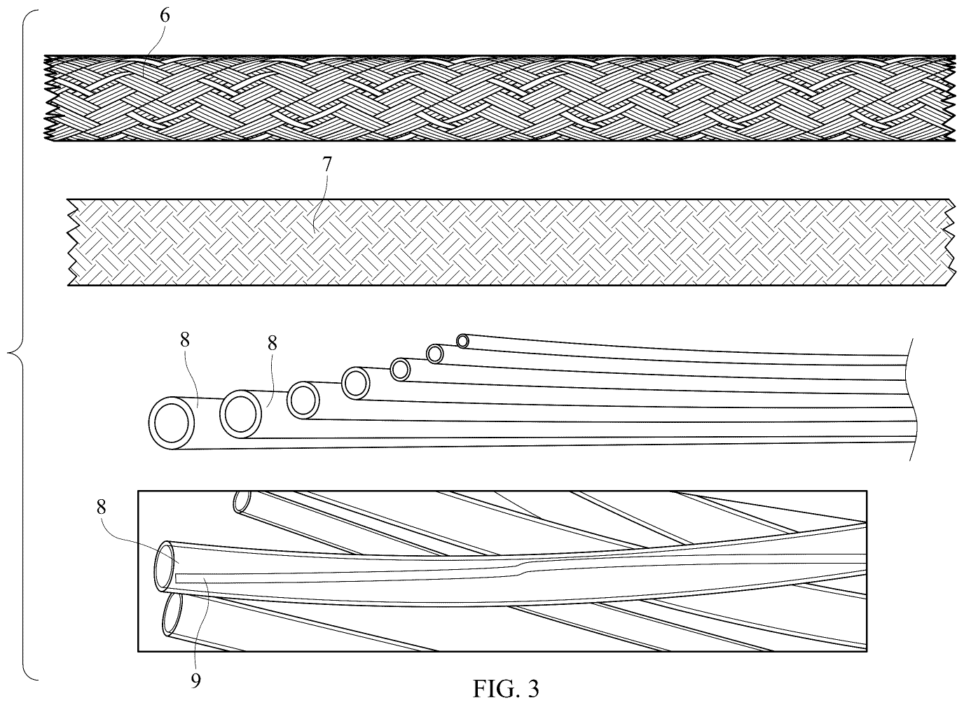

As depicted in FIG. 3, embodiments of devices of the present application may be formulated using any flexible materials that may be suitable for insertion into a human body. By way of non-limiting example embodiment, the devices of the present application may include Braided Composite Tubes (BCT) 6, which may include e.g., woven Nitinol and/or Polyethylene Terephthalate (PET) strips internal to the BCT, or Nitinol alone 7, e.g. internal to the BCT. According to example embodiments, the devices of the present application may include a material that stretches, such as an elastic material. For example, elastic polymer tubes 8 (e.g. including silicone) or other versions may be used. The devices of the present application may include one or more tubes 8 having the same or different diameters and/or thicknesses. Embodiments of the present application may be reinforced with metal 9 (e.g. metal strips) along the longitudinal axis to maintain flexibility, improve strength, and act as Xray markers. See FIG. 3.

Non-limiting examples of the present application include, for example, clastic polymer tubes (including, but not limited to, IV tubes) that are flexible, and may be optionally reinforced with metal along the longitudinal length of the tubes to maintain flexibility, improve strength, and act as Xray markers to allow for x-ray visibility. According to example embodiments, the metal inside the tubes will increase the strength overall to prevent breakage. By using metals of varying densities this will also stiffen up the cannula and can maintain a bend shape. This can assist by conforming to an anatomic region assisting in navigating.

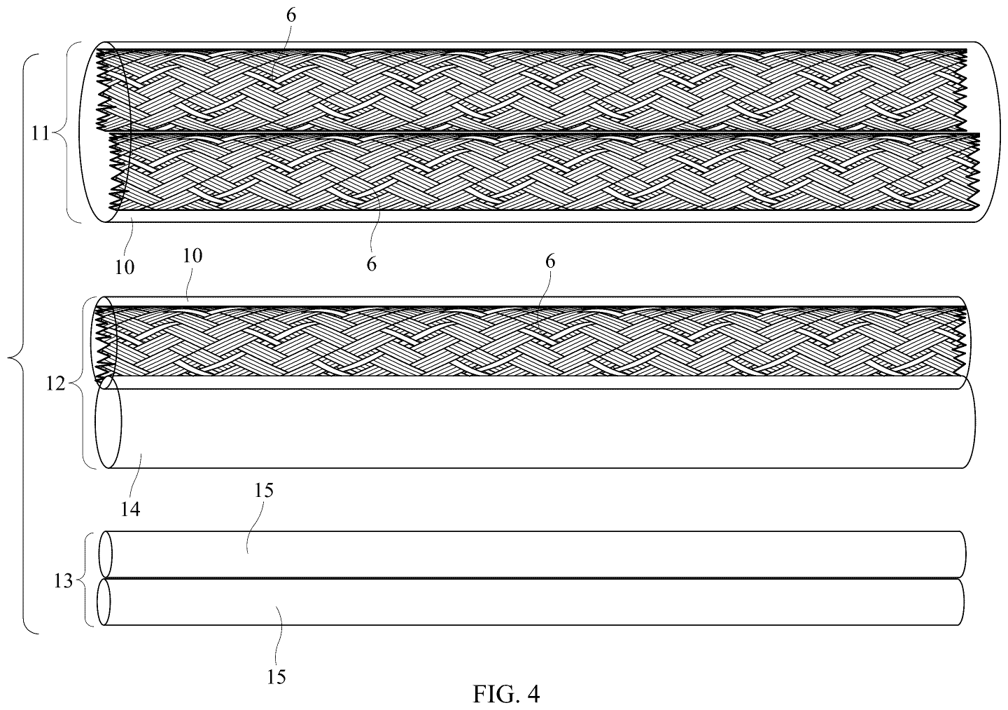

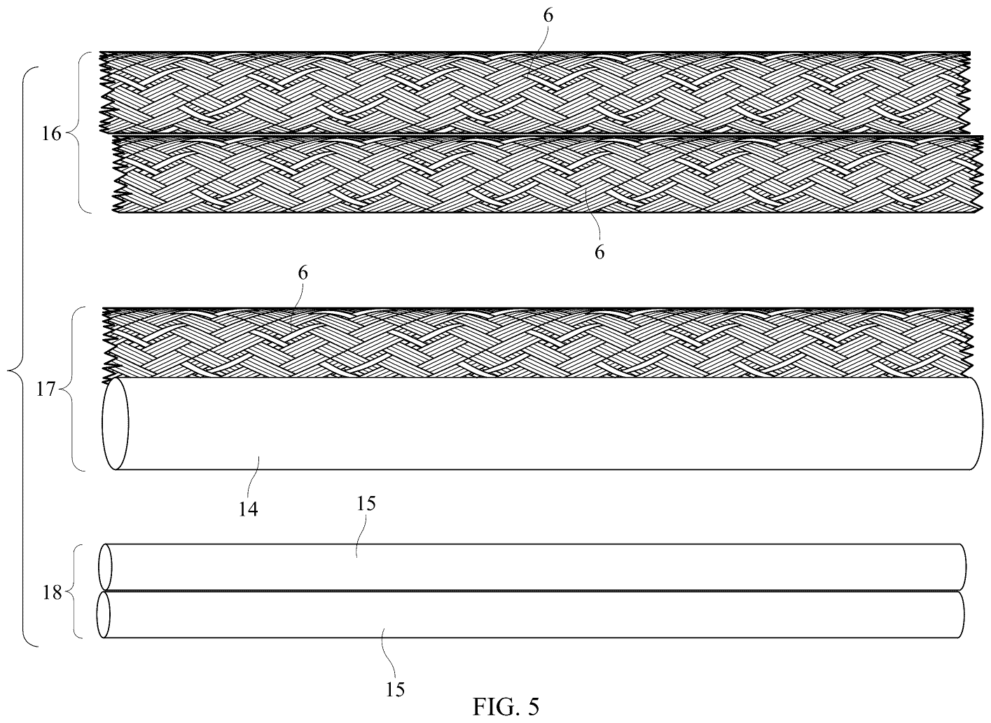

According to non-limiting example embodiments, in FASTER systems having multiple tubes, the tubes may include the same material as one another, or they may include different materials, which configuration may be determined depending on needs during surgery. As depicted for example in FIG. 4, some configurations according to example embodiments may include expandable tubes and/or minimally or non-expandable tubes. By way of non-limiting example embodiment 11, tubes may include a braided composite system (BCS) 6 with expandible Nitinol tubes inside. One or more BCS tubes may be coated in an elastic polymer 10. Other examples 12 may include a BCS tube 6 combined with an elastic polymer system 14. Other examples may include multiple elastopolymer systems 13 that include multiple elastomeric tubes 15. According to other embodiments, BCS systems may include Nitinol tubes inside a braided outer system 6 as shown for example in FIG. 5 as can be seen in embodiment 16. Other embodiments 17 and 18 may be similar to 12 and 13, but for example without an clastic polymer 10.

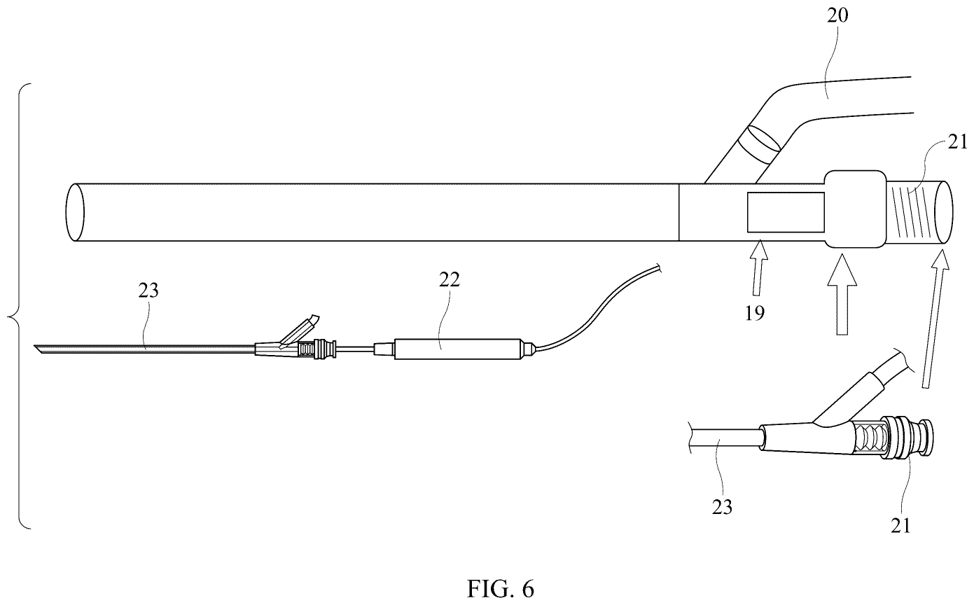

FIGS. 6-10 depict multiple examples of elastic polymer tubes in accordance with the present application, with an internal seal 19 and accessory IV tube 20. An IV tube is used as an example of clastic polymer tubes, to show the concept of the present application, but it should be understood that other flexible tubes made of various sizes, thicknesses, and materials may be used in accordance with the present application.

FIG. 6 shows that the example tube may be for example an IV tube 23, which is shown with a scope 22 inserted therein. The standard IV tube 23 may include an accessory inflow attachment. An end of the device may include a screw in 21 for additional inflow if not used as a scope portal.

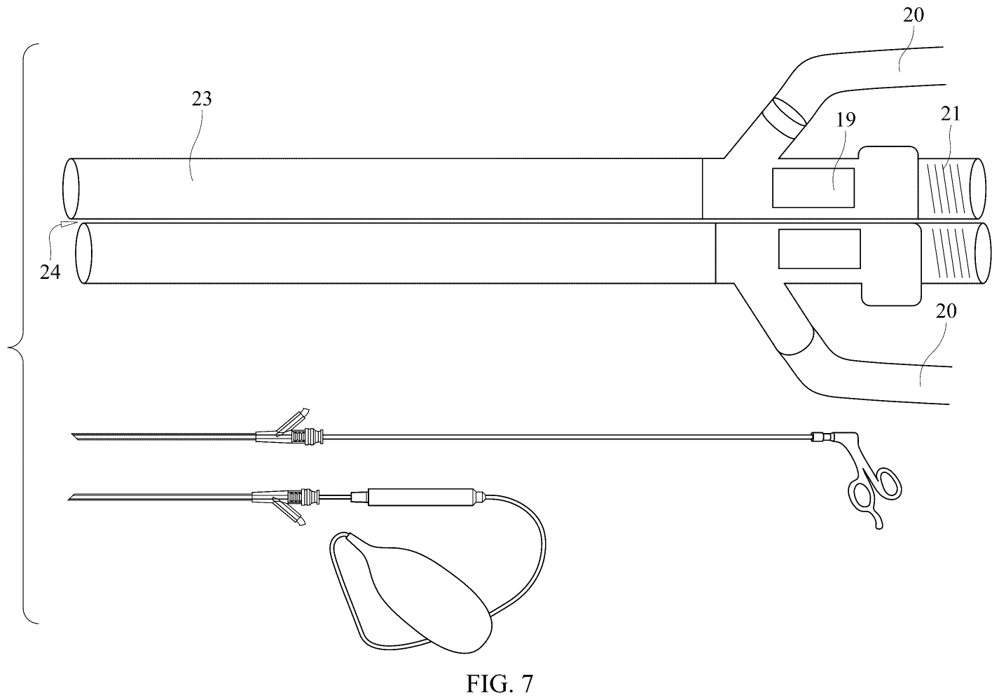

FIG. 7 depicts an example of elastic polymer tubes with an internal seal19 and an accessory IV tube 23. According to example embodiments, the tubes may have a guidewire tunnel 24 between tubes.

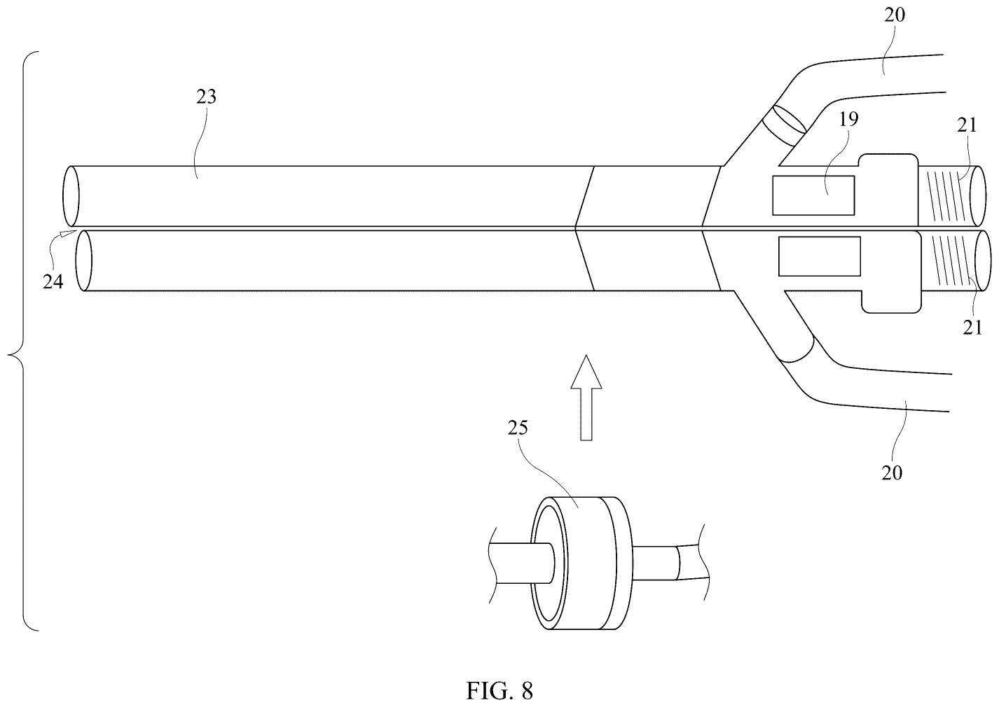

FIG. 8 depicts other examples of elastic polymer tubes 23 with an internal seal19 and an accessory IV tube 20. These embodiments include a round area 25 to allow one to rotate tubing.

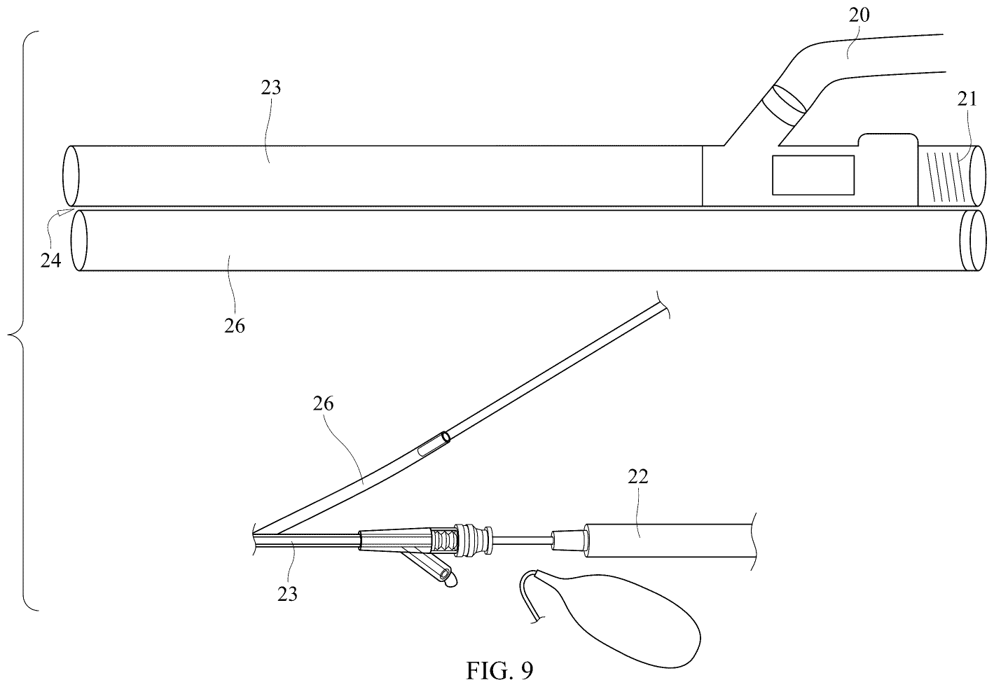

FIG. 9 depicts example polymer tubes 23 and 26 with internal seal and accessory IV tube 20. Tube 26 may be an additional portal for instruments, switching stick or scope passage, for example with no seal.

FIG. 10 depicts embodiments of the present application including an additional portal/tube 26 for instruments. In example embodiments, the tubes 23 and 26 may be interconnected by the same material and within the length of the tubes, reinforced with metal strips within the polymer. Multiple channels can also be used to increase the number of pathways for instruments. Shapes may vary by changing the size of one of the cannulas or using less portals.

FIG. 10 also depicts cross sections of various embodiments of the present application, which may include for example, two, three, (or more) tubes, having the same or different diameters. The cross sections include examples of cross sections of a first tube 27, second tube 28 and in some embodiments a third tube 29.

According to example embodiments, the present devices and systems can use one, two,

three, four or more tubes that are interconnected. They may be symmetric or asymmetric with varying size diameters. The working tube diameter can be based on surgical approach limitations and/or the instruments required.

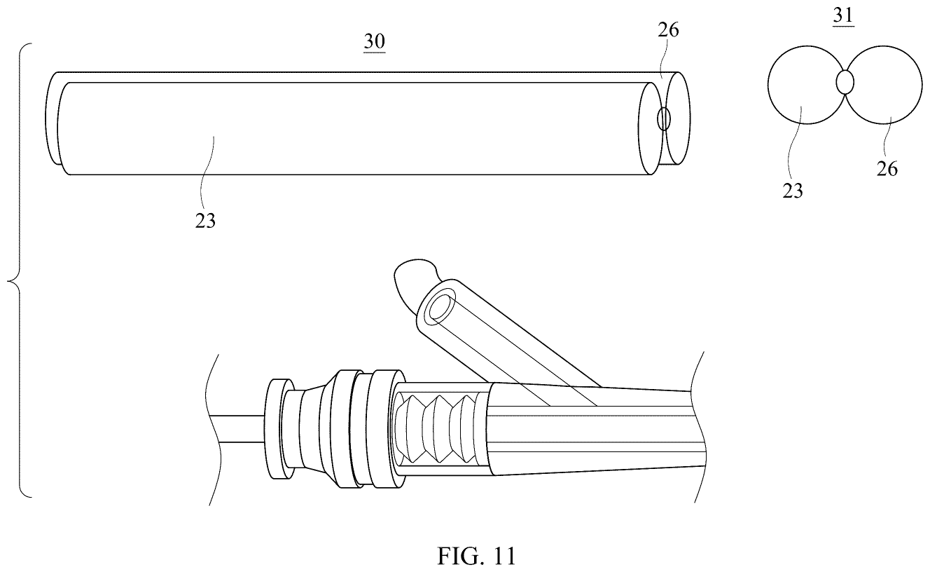

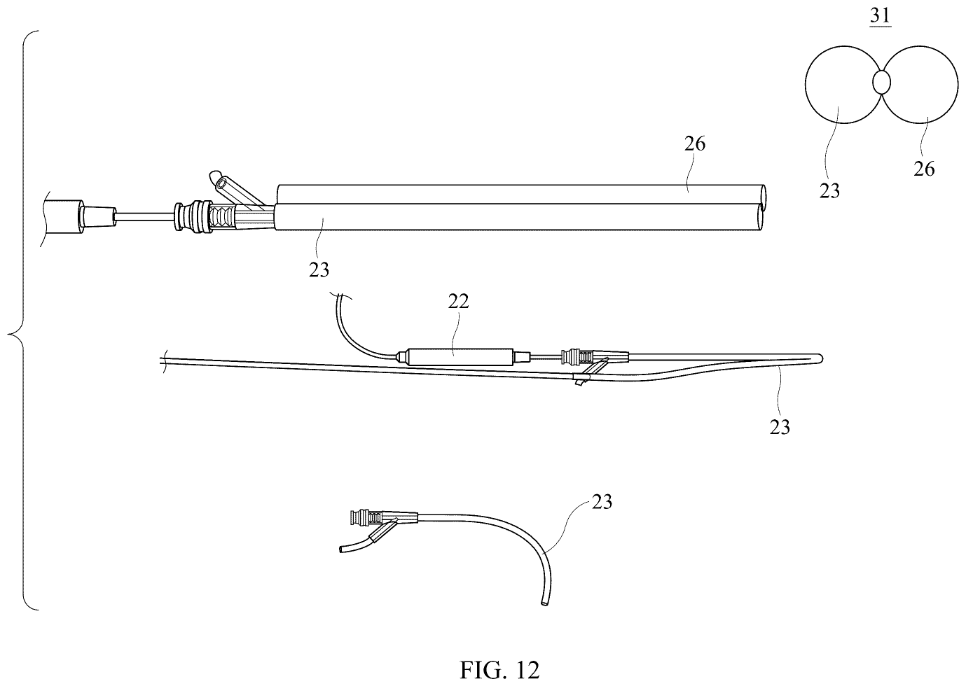

FIGS. 11-12 depict non-limiting examples of a bi-portal elastic polymer system, from a side perspective view 30 and from a cross section view 31. The diameters of the two tubes can be the same or different diameters and the tubes may be the same or different materials. FIGS. 11-12 also depict examples of a bi-portal (two interconnected tube) system according to the present application, put into use, for example using IV tubing. The diameter may be selected depending on which instrument is being used to allow instrument passage, and to prevent backflow. According to FIG. 12, a seal may be provided, so the instrument does not slide in and out of the tube. FIG. 12 also shows an example in which the two tubes are interconnected for the length of the structure. It is contemplated however, that there may be a portion of the present devices (for example at one or both of the ends), 'where the tubes are not connected for a portion of the device and/or in which a tube of the system may be longer than the other(s). The non-limiting depicted example also includes metal through the length of the device, to avoid breakage.

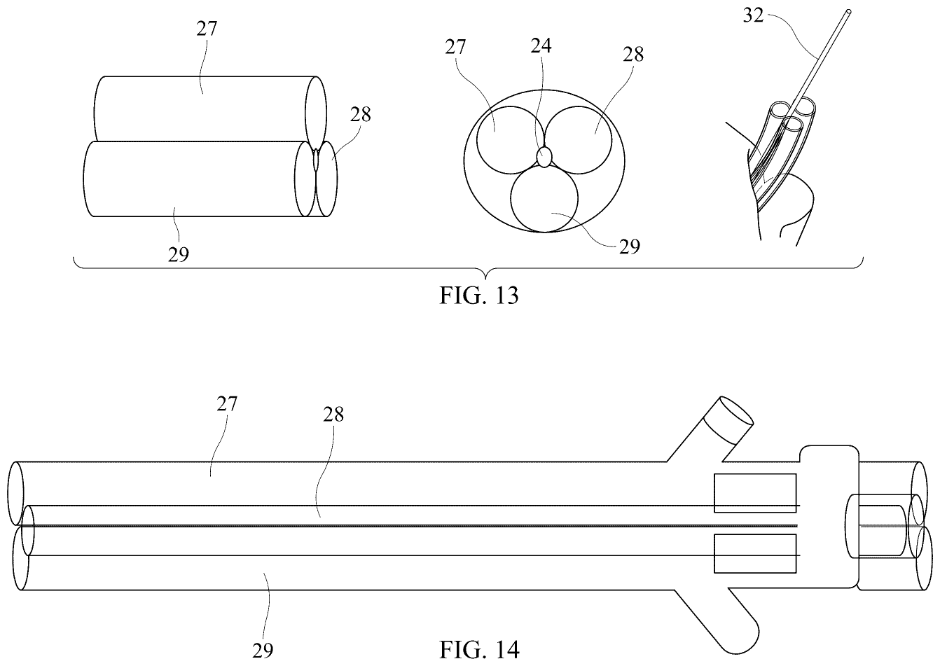

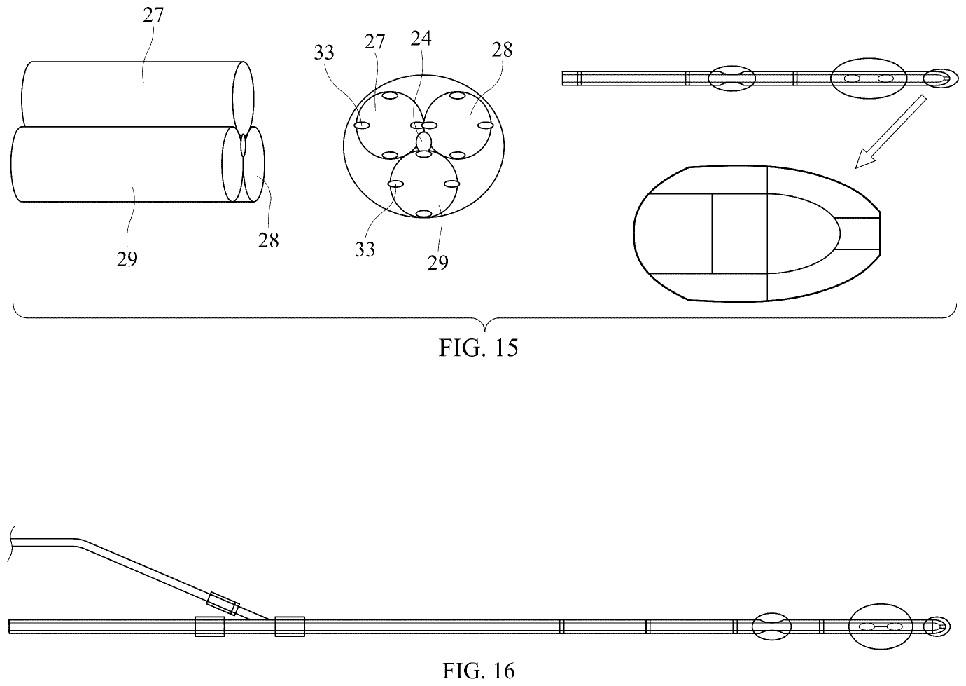

FIGS. 13-15 show non-limiting examples of tri-tube embodiments of the present application. According to example embodiments, the depicted embodiments may include an clastic polymer, but as discussed herein, are not limited to that material. As with other embodiments herein, the tube diameters may be equal or varying diameters and may be made of the same or different materials. As shown in FIG. 13, a guidewire 32 may be included in, or used with, the present devices. The guidewire 32 can be passed through the middle of the first, second and third tubes of the present application and can serve as a switching stick to swap e.g., uni-portal/bi-portal/tri-portal or multi-portal options easily during a procedure. As shown in FIGS. 13 and 14, in a tri-tube system of the present application, the first 27, second 28 and third 29 tubes may be interconnected to one another while leaving open a space in the middle 24 of the three tubes (a guidewire tunnel). The guidewire 32 may be a separate component from one or more reinforcing metals 33 that may be used to strengthen the present device and potentially act as X-ray markers (see FIG. 15). FIG. 14 (and other figures) show that the present devices may include one or more IV accessory tubes.

The FASTER systems and devices herein have at least the following advantages when compared to rigid tubular scopes and cannulas, typically used for surgery. FASTER has the advantages of reducing the outer diameter (OD) required to house all the necessary items to perform a surgical procedure arthroscopically/endoscopically. These include the working cannula, camera cannula, fluid (Inflow/Outflow) and/or gas cannula, and/or the suction cannula. In embodiments, because the shape is not round, this reduces the overall surface area in contact with surrounding tissue, which may be especially important when working around nerves.

The FASTER system can conform to the optimal surgical site diameter instead of always using a fixed OD opening tube. This is because of the properties of the material used in the tubes. Example tubes will conform around a surgical instrument and are strong enough to tolerate instrument passage because they may have metal strips throughout the length of the tube.

Another advantage of these FASTER systems is that they are strong, flexible, collapsible tubes and/or sleeves that allow the passage of instruments of varying sizes and shapes without compromising the outer diameter (OD) constraint issues that limit surgical access.

The FASTER system will conform to distinct size instruments without significantly increasing the (OD) diameter when compared to rigid scopes.

The FASTER system will collaborate with multiple company products and not require their modification at all, allowing them to function as advertised but with better versatility/utility.

The FASTER system can be sleeved Bi-tubular or Tri-tubular, or further multi-tubular and attaches instruments together. These include attaching the endoscope directly to an instrument. By attaching the endoscope directly to the instrument, this allows the two devices to move together without an additional access cannula. This omits the need for additional working cannulas.

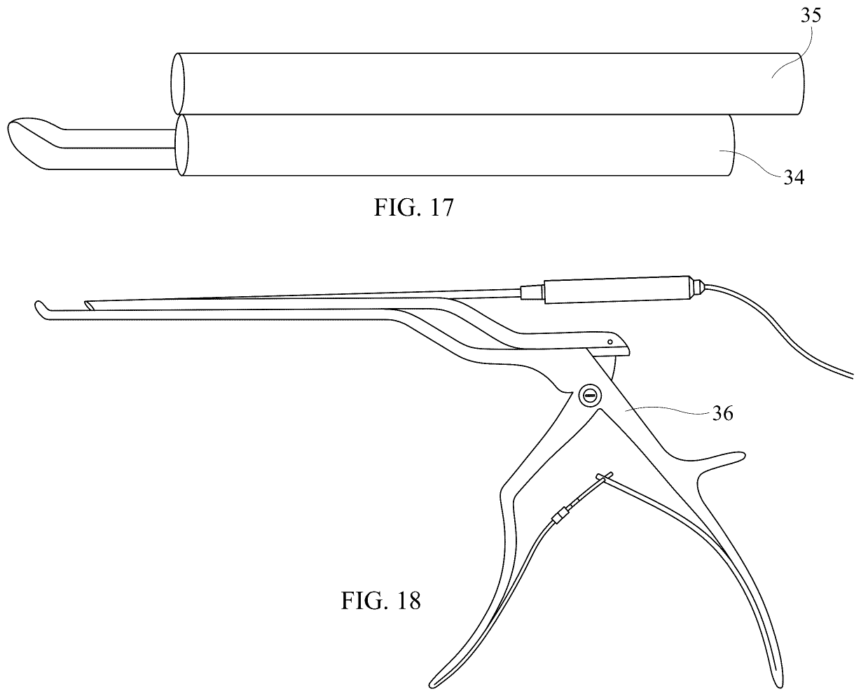

In example embodiments, a FASTER sleeve with two stretchable tubes can slide tightly over a standard Kerrison used during spinal surgery. When an endoscope is placed within the second sleeve on the top area of the Kerrison it improves the device's utility. The instrument (within the present FASTER system), is now a Kerrison that has improved visualization via the endoscope pointed at the jaws of the device. When the Kerrison is advanced out of the surgeon's typical field of view, attention is turned to the endoscopic view on the monitor so the surgeon can see the structures out of their view. This can be used in closed spaces during the opening of minimally invasive surgery. As shown in FIG. 18, the present devices provide a system in which a camera portal can be pointed at the working “jaws” providing better visualization of the Kerrison, which allows access to areas not seen well with the naked eye. The present devices also allow the surgeon to use a larger instrument without a separate incision (portal) and without the constraints of a working cannula. The Kerrison can enter the surgical arca without using a portal and this enhances maneuverability, as it is not restricted by a rigid cannula.

Sleeves can also be placed over cautery, pituitaries, burrs or other surgical tools typically used during surgery. The sleeves may have (one or multiple) cinching lasso tightening device(s) to prevent the sleeve from telescoping on the instrument.

The FASTER devices of the present application can eliminate the need for a working channel by attaching the scope/inflow cannula to the instrument directly, by cach being in connected tubes of the present devices. This allows the instruments and camera to move together in symbiosis similarly to the cannula without the disadvantages of the rigid cannula. When instruments are linked together with the present devices and systems, this improves their overall ability to work and function.

The FASTER system improves instrument maneuverability, as there are no fixed tubes, only flexible ones that do not limit movements: Because the tubes are flexible throughout their length. Thus, the present system can maneuver and get around corners within the patient, which cannot be done with previous devices.

The FASTER system can be deployed to varying diameters and lengths and this will not affect its flexibility, bendability, or its ability to acceptable larger and/or curved instruments. Fixed-curved instruments are typically an issue for long rigid tubes that's shape can't conform. The FASTER system stretches to accommodate instruments of varying sizes, curves, shapes and lengths without issue.

The FASTER system can be introduced in a small/narrow form, and then a Braid Nitinol or Bare Nitinol tube can be deployed/expanded, similarly to a vascular stent. The FASTER system can be driven to areas of the spine the would be impossible and/or difficult to reach percutaneously with a rigid endoscope. Once the FASTER expandible system is deployed, both flexible and rigid instruments of varying diameters can be passed through the tubes to perform a multitude of surgical procedures that were not previously possible.

The FASTER cannulas can be deployed, standalone, or also be deployed around an endoscope and other necessary cannulas inside of the body.

Any access cannulas can also be linked together. Two standard cannulas can specifically be joined with sleeves connecting the endoscope (camera/inflow) and instrument directly and/or a tunnel for device passage. This eliminates the need for restrictive rigid cannulas but maintains the advantage of linking the camera and its field of view to the “working” instruments.

The FASTER system can include multiple tubes that are attached to the endoscopic camera. Because there are multiple expandible tubes attached to the camera, one can use multiple instruments at the same time. For example, a three cannula FASTER system has cannulas that could be use simultaneously to house a cautery device, a pituitary, and the Endoscope/Inflow/Outflow: This allows all three instruments to move together in symbiosis. This eliminates the need to pass instruments in and out of one working channel, reducing surgical time.

Methods of using a flexible Braided Composite/Nitinol tubes improve the function of standard surgical instruments. By way of example, the FASTER sleeves may be used to attach a “chip on a stick” small endoscope to a Kerrison's instrument to expand visualization of the working tip of the device. This expands the field of view allowing the surgeon to see areas outside the surgical fields view.

The FASTER devices and system will overcome many of the shortcomings of using fixed tubular retractors, cannulas and/or working channel endoscopes currently used in surgery. These devices will also allow smaller endoscopes to be linked and attached to multiple instruments. This will allow the endoscope and instruments to work in harmony without increasing the outer diameter or using a rigid cannula which has many limitations. Any access cannulas can be linked together; specifically joined with an endoscope (camera/inflow tube) and varying instruments eliminating the need for restrictive rigid cannulas.

The FASTER system creates a novel system and pathway for instruments to be introduced seamlessly from the external skin to area of pathology for surgical repair. The FASTER system can be deployed or driven to areas of the body that would be difficult or impossible to reach via a percutaneous technique with a rigid scope.

After the FASTER system is deployed and/or attached to a surgical device, it will improve the functionality and the instrument/scopes' ability to perform a surgical task. Both flexible and rigid instruments of varying diameters can be passed through the FASTER system to perform a multitude of surgical procedures because the endoscope is linked together with stretchable tube portion of the system.

The FASTER devices or system can include multiple interconnected tubular sleeves, deployed as standalone working cannular (from the skin to the pathologic area), or can be deployed after the scope is placed within the body. Multiple access cannulas can also be deployed, so that the endoscopic camera is attached to multiple working instruments: such as, but not limited to, a cautery device, motorized burr, shavers, and biters/pituitary instruments can be delivered accurately through the tissue.

Embodiments of devices of the present application may include Flexible Access Supplementation Tubes for Endoscopic (Exposure and) Retraction. The devices/tubes of the present application may include a one or more flexible tubular sleeves. According to example embodiments, the present application includes multiple interconnected tubular sleeves. The interconnected sleeves may be single tubes that are thereafter connected to one another, or the device may be fabricated as a single device that has interconnected tubular sleeves. FIG. 16 depicts an example from a fabrication company that makes catheters, and illustrates options available for elastic polymer tubes.

FIG. 17 depicts a bi-portal interconnected FASTER system in which the tubes are made of multiple materials, depending on their use. In this example, the system includes an elastic polymer adherent sleeve that will slide and be secured over existing instruments to increase the utility of the device during open or endoscopic surgery. Interconnected to the elastic polymer sleeve is a deployable Nitinol Tube (or BCS), that can be an expandable accessory tube.

During minimally invasive spinal surgery via a Wiltze Spine approach, contralateral decompression of the spinal canal can be performed under better visualization as the endoscope as a supplement. This can be used to see the working area of the device when the naked eye cannot see under the lamina. This will allow you to decompress tissue, bone or disc material causing stenosis in the spinal canal.

In FIG. 17, a first tube 34 may be an elastic polymer adherent sleeve that is attached to a stretchable sheath. A second tube 35 may be a deployable nitinol tube (or BCS) that when deploys, expands accessory tube and polymer thickness limits expansion similarly to a vessel providing resistance to the nitinols pre-shape once the catheter sheet is removed.

FIG. 18 depicts an instrument (scope 36) that may be capable of working together with examples of the present FASTER system. The present systems improve existing instruments and technologies capability of working together in a symbiotic fashion. When two instruments are linked together with the present devices, this improves their overall ability to work and function. Together the camera portal pointed at the working “jaws” provides better visualization for the Kerrison, allowing it to access areas not seen well with the naked eye. The device enhances the utility of the scope because it allows the surgeon to use larger instruments without a separate incision (portal) and without the constraints of a working cannula. The Kerrison can enter the surgical arca without using a portion and this enhances maneuverability as it is not restricted by a right cannula.

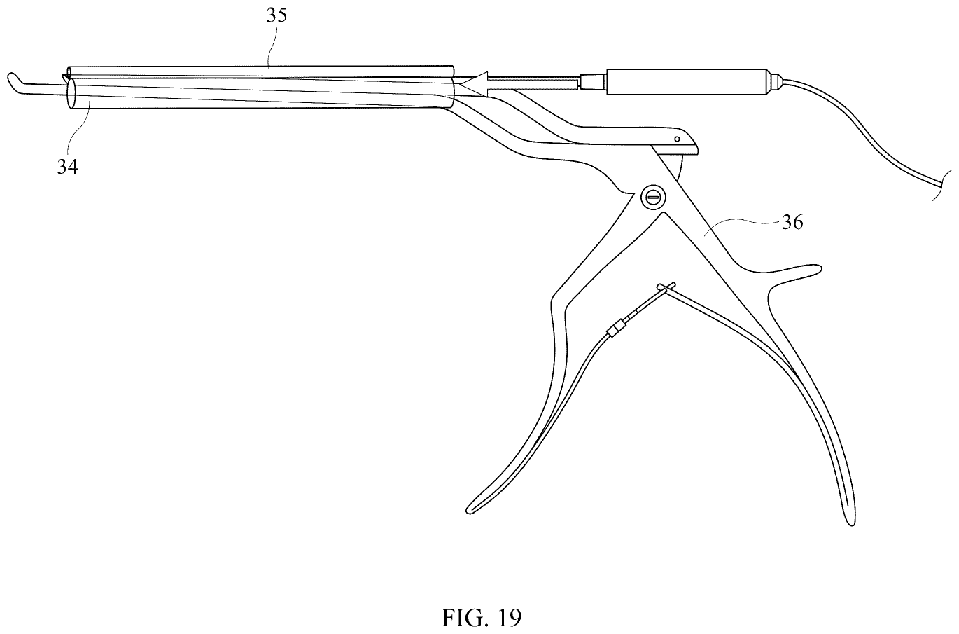

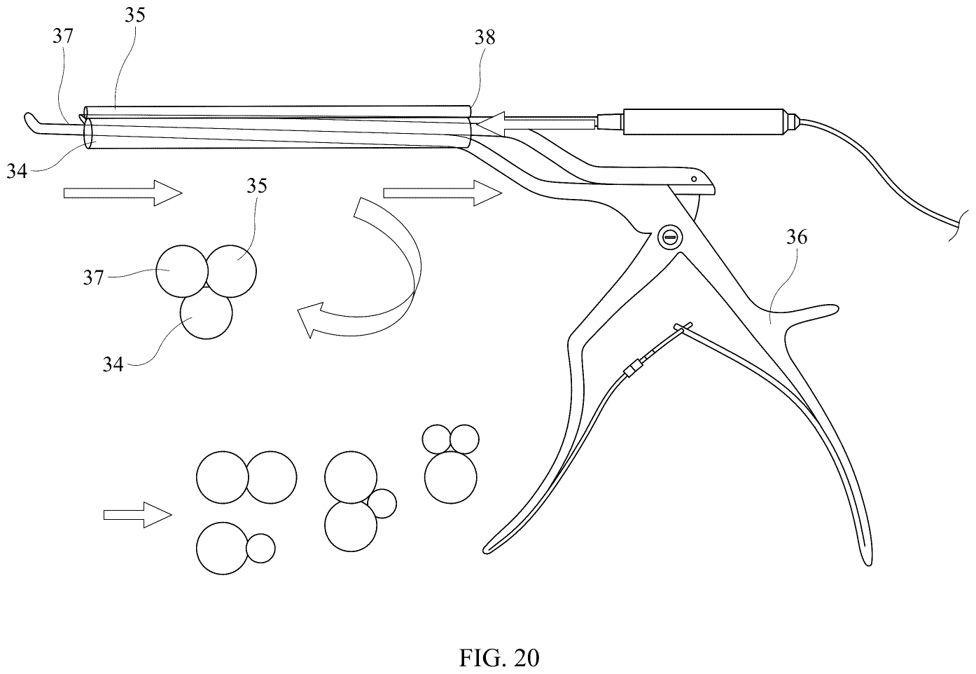

FIGS. 19-20 depict non-limiting example embodiments of the present application, which include interconnected tubes 34 and 35, in which at least one of the tubes 34 is sized and configured to slide onto an instrument 36 (the instrument may be in closed configuration), and at least one additional interconnected tube 35 is available as an accessory channel. In these embodiments, the device may be for example bi-portal or tri-portal and the tubes may be interconnected by the same material and they may be reinforced with metal within the length of the tubes. In example embodiments tubes may be interconnected of the same material with metal stainless steel or nitinol traveling the length of the tube to improve strength, but maintain flexibility.

FIG. 20 shows interconnected flexible tubes in accordance with example embodiments, that slide onto a closed instrument 36. Tubes may be interconnected of the same material and within the length of all tubes reinforced with metal. Multiple channels can also be used to increase the number of pathways for instruments. As shown at the bottom of FIG. 20, shapes may vary by changing the size of one of the cannulas, or by using fewer portals. Example embodiments may have inflow outflow seals to reduce fluid flow at 38.

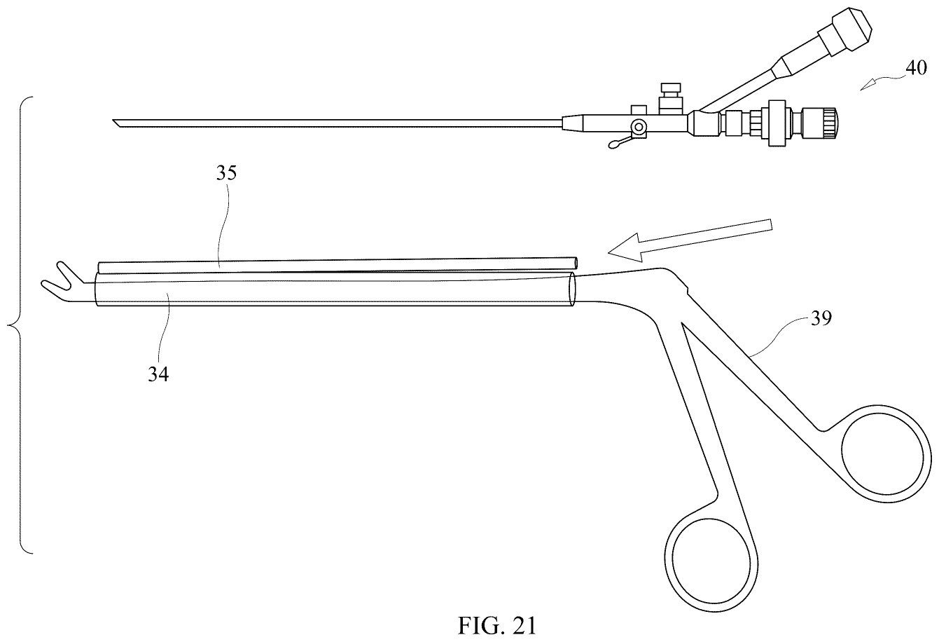

FIG. 21 also shows embodiments in which one of the sheaths/tubes 34 slides over an initial instrument 39, and then another (or multiple) interconnected sheath/tube/accessory channel 35 is available for, e.g., an arthroscope (e.g. “chip on a stick” scope 40) or other instrument to be inserted so the second device or scope is interconnected to the first. The interconnected sheath may expand to house other instruments in the working channel.

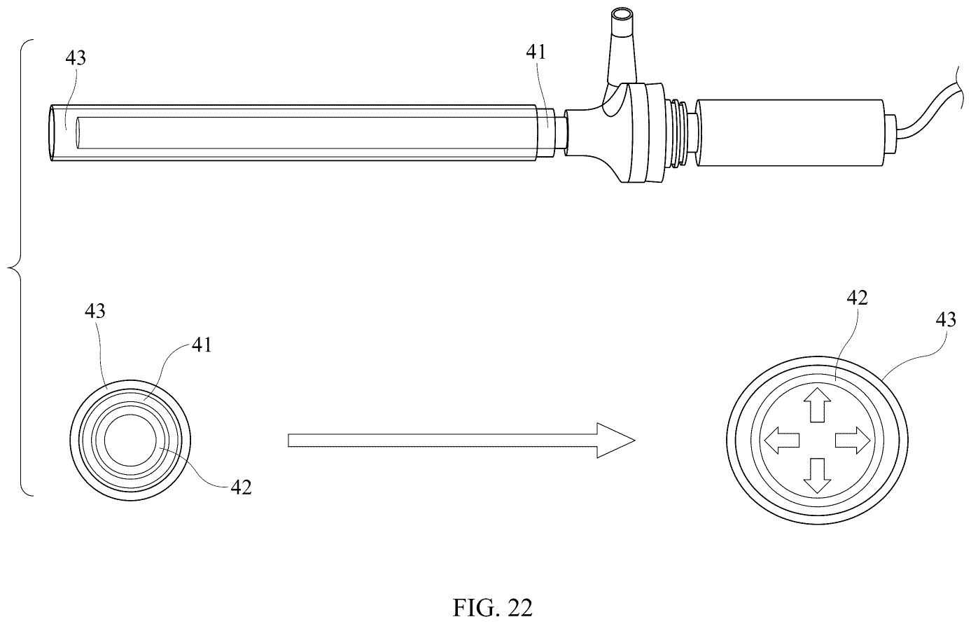

FIG. 22 depicts embodiments including a Nitinol tube\\ and showing how an additional removable catheter sheath 41 may be included in the original device to maintain a Nitinol tube 42 in a closed position until it is desired to deploy the Nitinol tube 42 into an expanded position (for example after insertion to a desired location in a patient). After the removable catheter sheath 41 is pulled back, the Nitinol tube 42 is expanded within an elastic polymer tube 43 and functions as a working channel for instruments and endoscopes. Other instruments can be placed through a larger portal because the inner diameter is expanded.

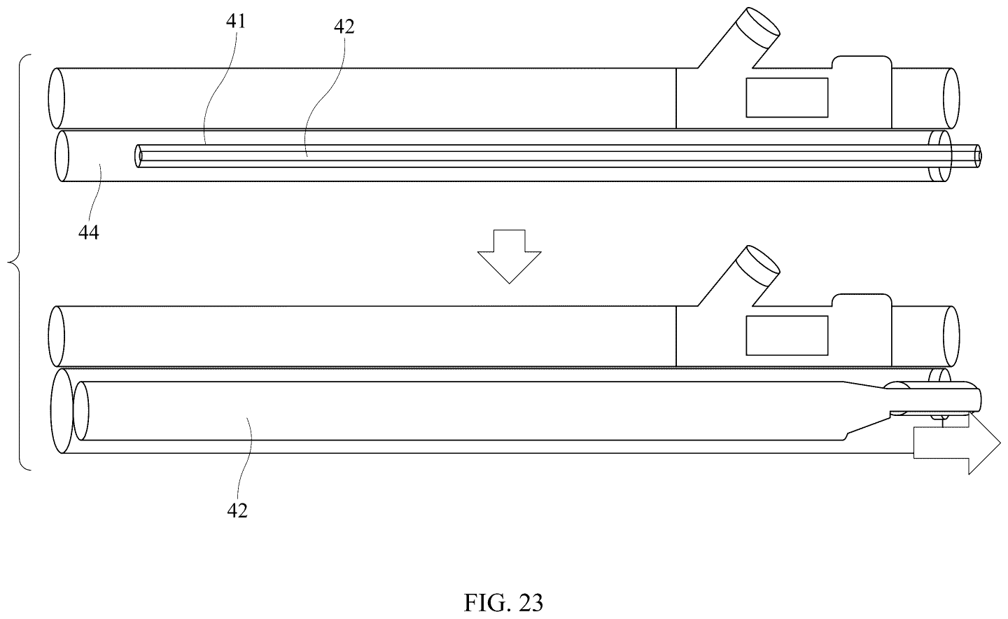

FIG. 23 depicts potential modifications of embodiments of the present application, which may include for example an expandible Nitinol material. In FIG. 23, a removable catheter sheath 41 is holding a compressed stent 42 in the upper portion of the figure. The sheath 41 can be inserted into the tube 44 and then the sheath 41 removed, thus allowing the expandible nitinol stent 42 to expand (see bottom portion of FIG. 23). This may result in the cannula becoming permanently larger.

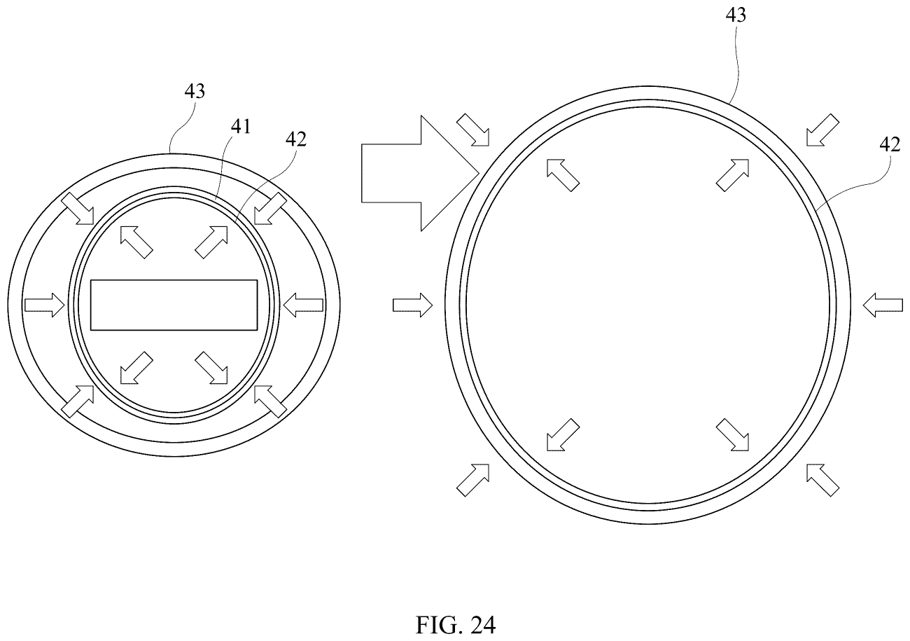

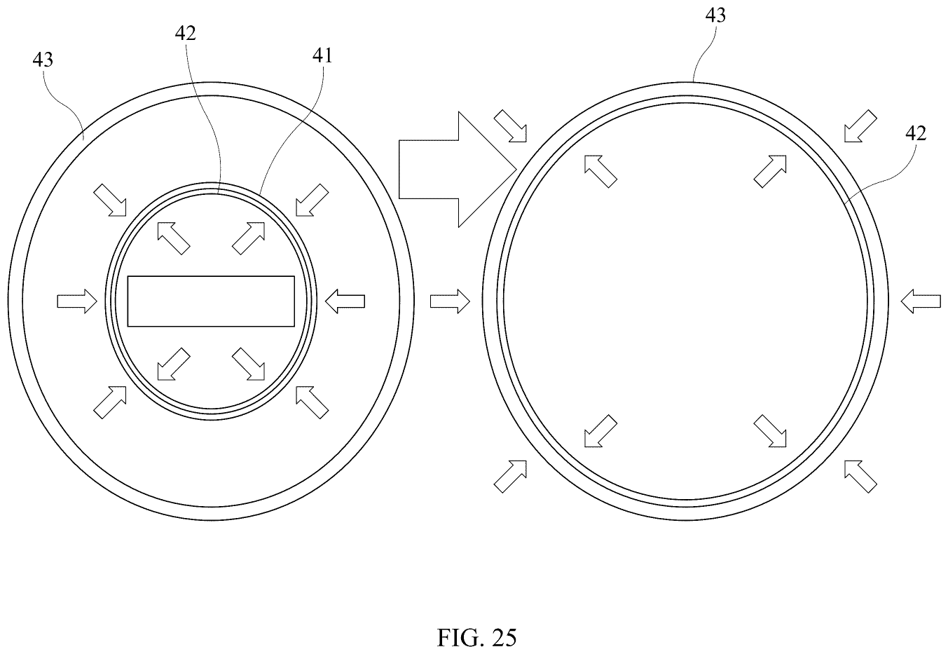

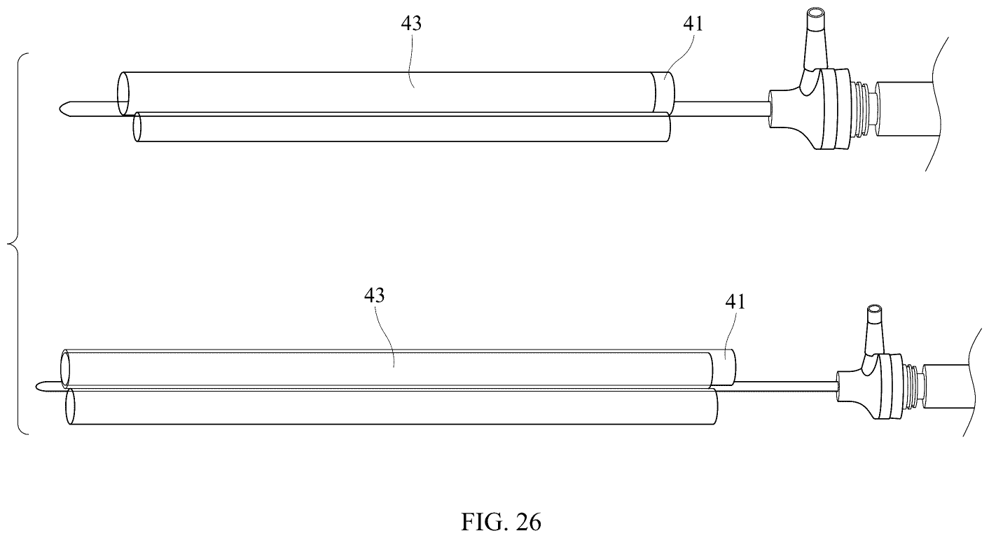

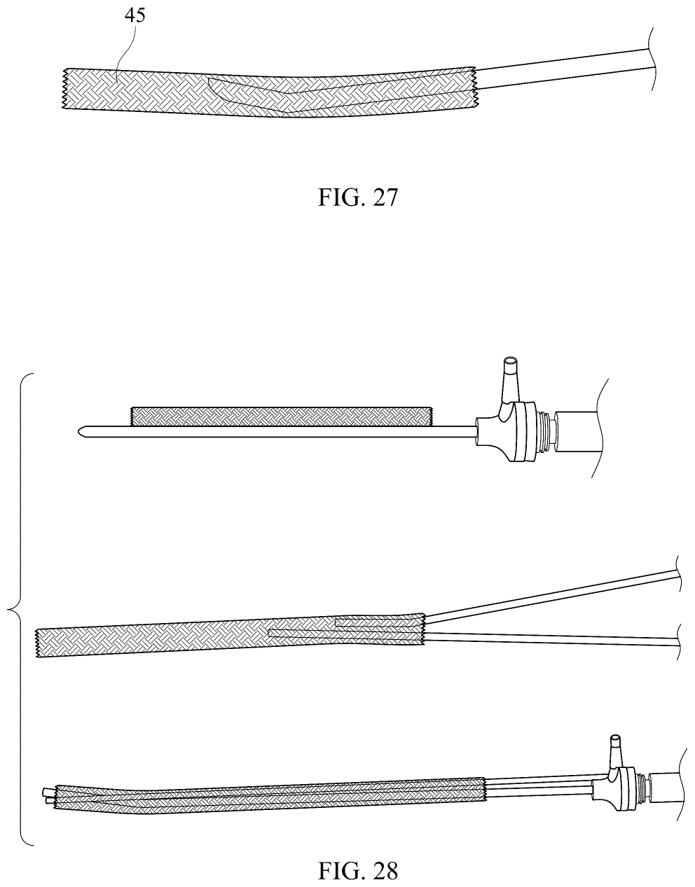

FIGS. 24-25 also show a cross-sectional view of how Nitinol 42 may be used in embodiments of the present application, for example being introduced in a compressed form with a removable sheath 41 around it. When the removable sheath 41 is removed, and the Nitinol tube 42 expands, a working channel is created, with an outer tube 43 (such as a smooth tube) to make it easier to move the tube through a patient, and to restrict the maximum shape deployment of the Nitinol tube. The shape, size and material of the outer tube may be selected based on desired size and use. FIGS. 26-28 show examples of the present application in use, in which an expandable Nitinol tube is used. FIG. 27 shows an example of how the addition of nitinol expandible can accept curved instruments. FIG. 28 shows three examples of nitinol metal tube stretchability and stretch, which would increase the size of the design to increase portal inner diameter without compromising strength.

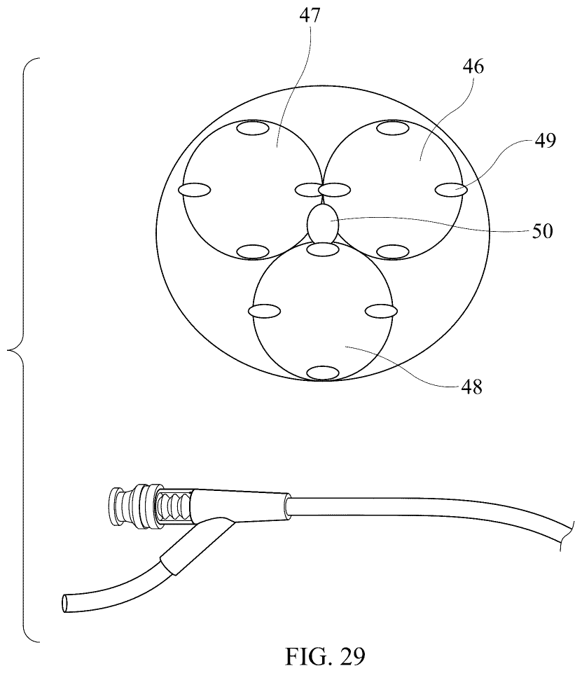

FIG. 29 depicts non-limiting examples of the present FASTER tubes. In particular, a cross section of a tri-tube embodiment is shown in which the tubes 46, 47 and 48 are one piece of material fused together in the middle 50. One or more of the tubes can be expandible with or without nitinol/braided composite catheter insertion. The tubes can have standard IV type Luer locks for fluid inflow and outflow to optimize visualization. Example tubes may have reinforcing materials 49.

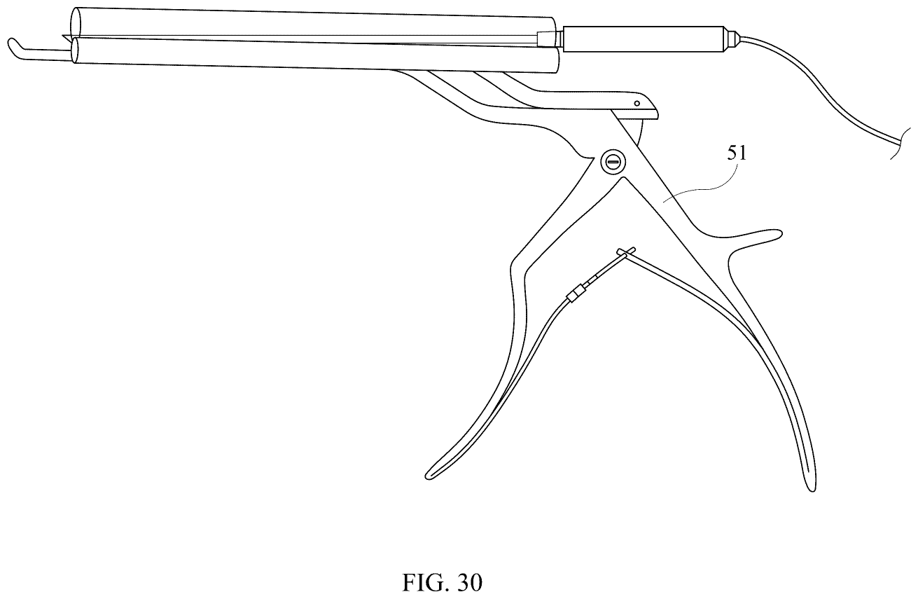

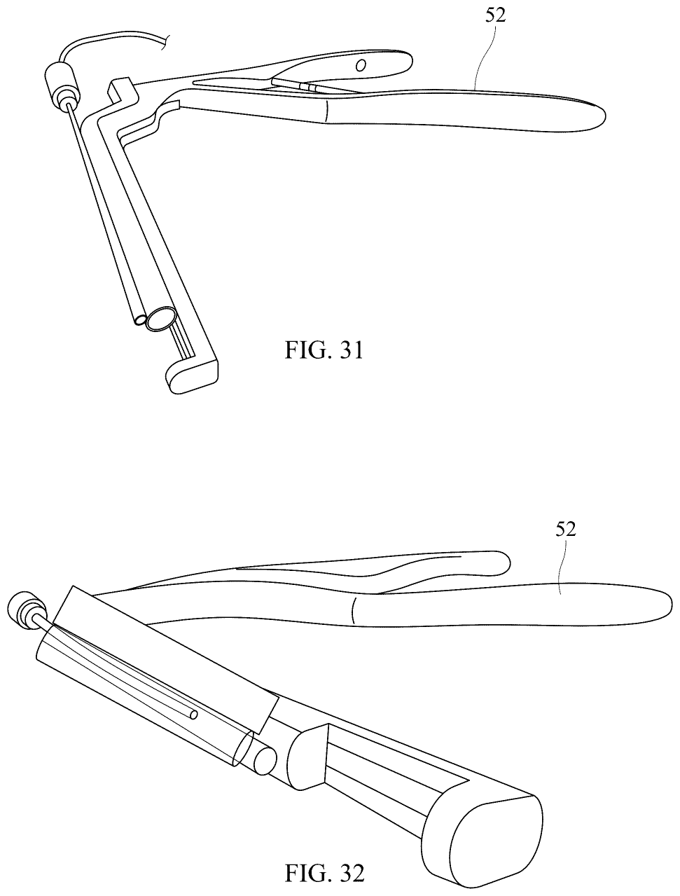

FIGS. 30-32 (and 19) depict examples of a Kerrison 51and 52 with the present FASTER system attached (and not attached—see FIG. 31).

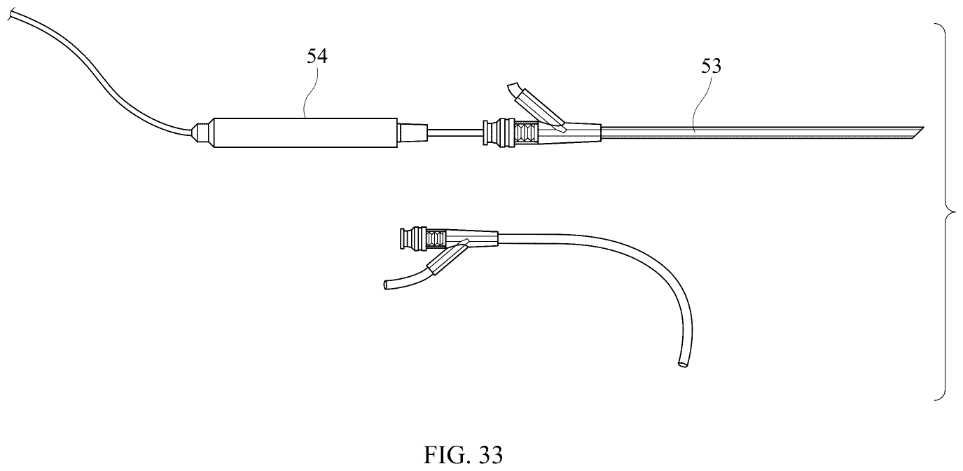

FIGS. 33 and 2 depict examples of passing a scope 54 within IV tubing 53 and an IV tubing system with a second instrument to depict the flexibility the present application may provide.

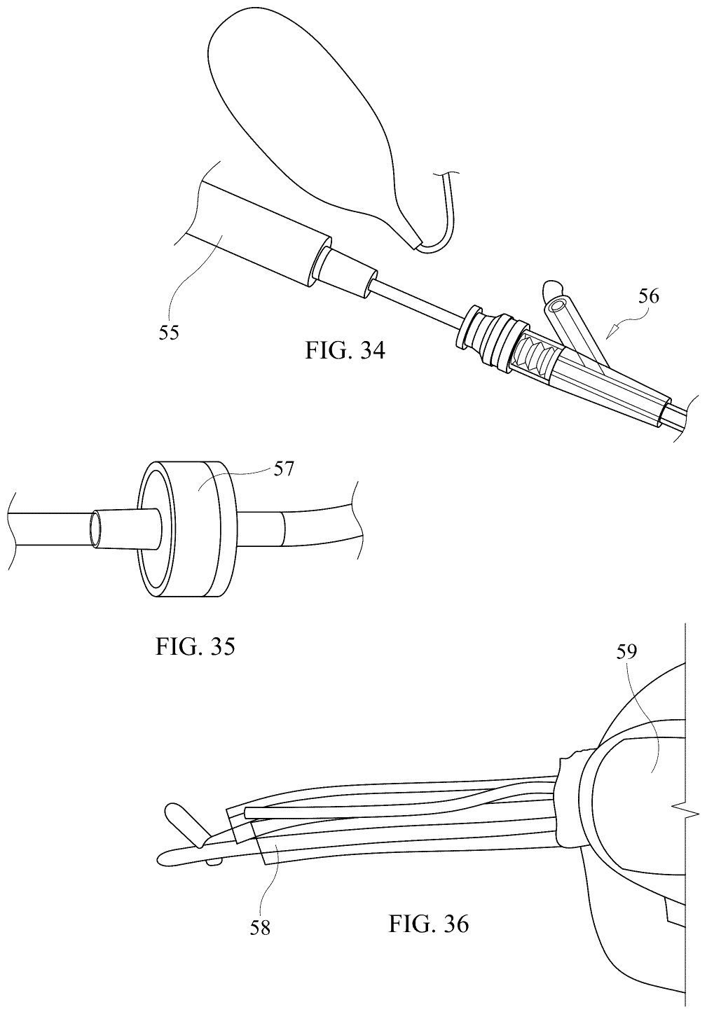

FIG. 34 shows an example of a scope 55 and secondary fluid channel 56 incorporated into the tube.

FIG. 35 shows a plastic round holder 57 that can be used to turn and spin the interconnected tubes of the present application. This holder may be made of different materials and is an optional component of embodiments of the present systems and devices.

As depicted in FIG. 36, the present system may be relatively small in size. For size reference, an indication of roughly the size of a thumb 59 is shown near a system or device of tubes 58 in accordance with embodiments of the invention.

According to non-limiting embodiments, the tubes can have standard IV type Luer locks for fluid inflow and outflow to optimize visualization.

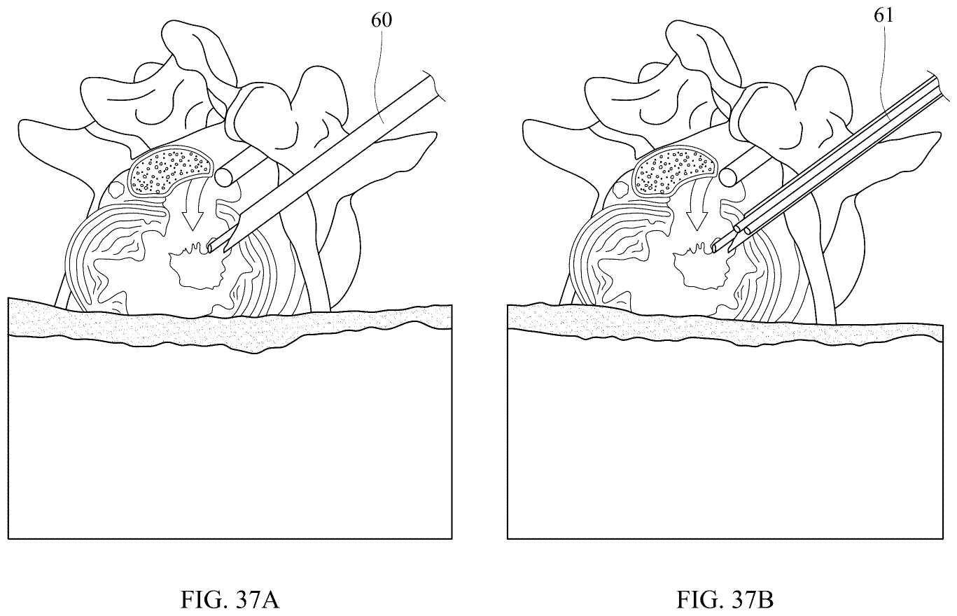

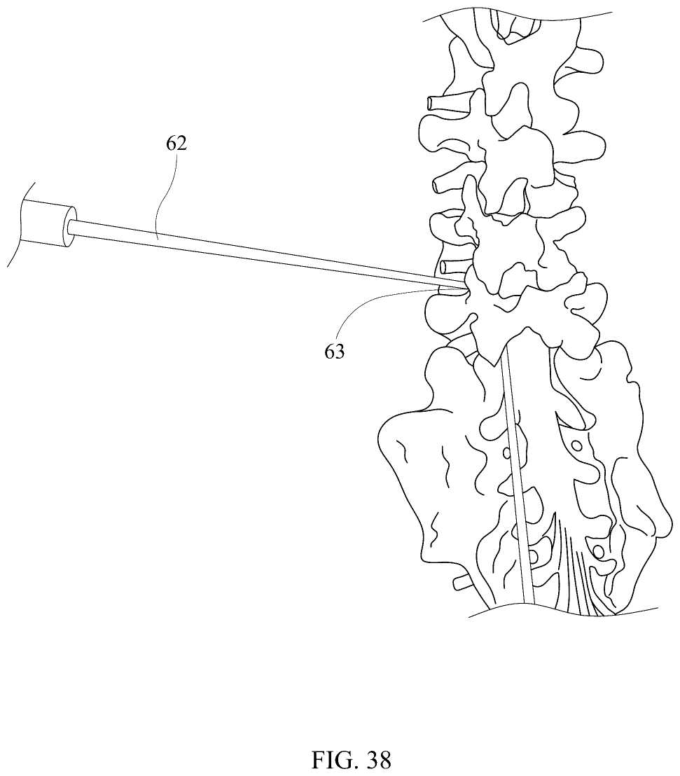

FIGS. 37A and 37B depict example embodiments of the present FASTER system (61 of FIG. 37B on the right) vs. a traditional rigid scope on the left (60 of FIG. 37A on the left). The FASTER system 61 includes smaller tubes over an initial dilator or guidewire, which can be exchanged for the scope. The tubes are flexible and conform to the size of the instruments used. Because the tubes are flexible, this allows better maneuverability than prior tubes 60 and because the tubes conform, they reduce the surface area of the device in contact with surrounding structures within a patient.

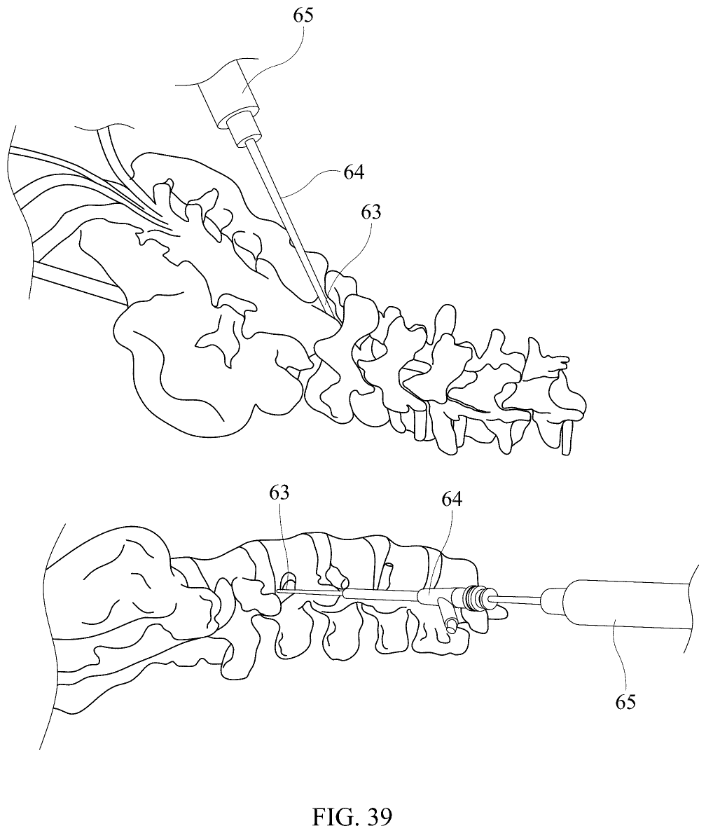

FIGS. 38 and 39 compare the use of an endoscope in a standard tube (62 of FIG. 38) vs. an endoscope 65 of FIG. 39 in a plastic tube 64, such as an elastomer medical tube, which allows for more flexibility in delivering devices to a desired location 63 of a patient.

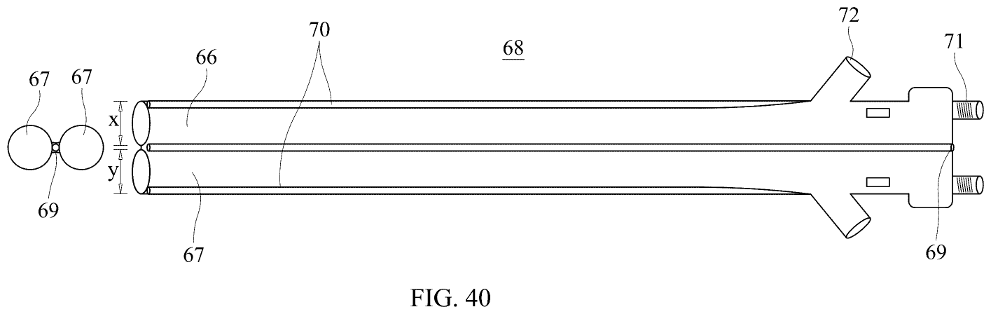

FIG. 40 shows a non-limiting embodiment of an endoscope 68 in accordance with an embodiment with a standard typical medical tube/cannula of a FASTER device/system of the present application. In the depicted example, the flexible interconnected tubes 66 and 67 may have an inner diameter x and y of e.g. 1.9-2.5 mm each (or 1.7-3.0 mm each), and can be of varying lengths, depending on the instruments/scopes that need to be accommodated for a particular procedure. The device may have a guidewire hole or channel 69. in example embodiments, there is also at least one built in irrigation tunnel 70. Irrigation can be attached or screwed in for example at 71 or 72 (or the channel(s) 70 may act as working instrument channels).

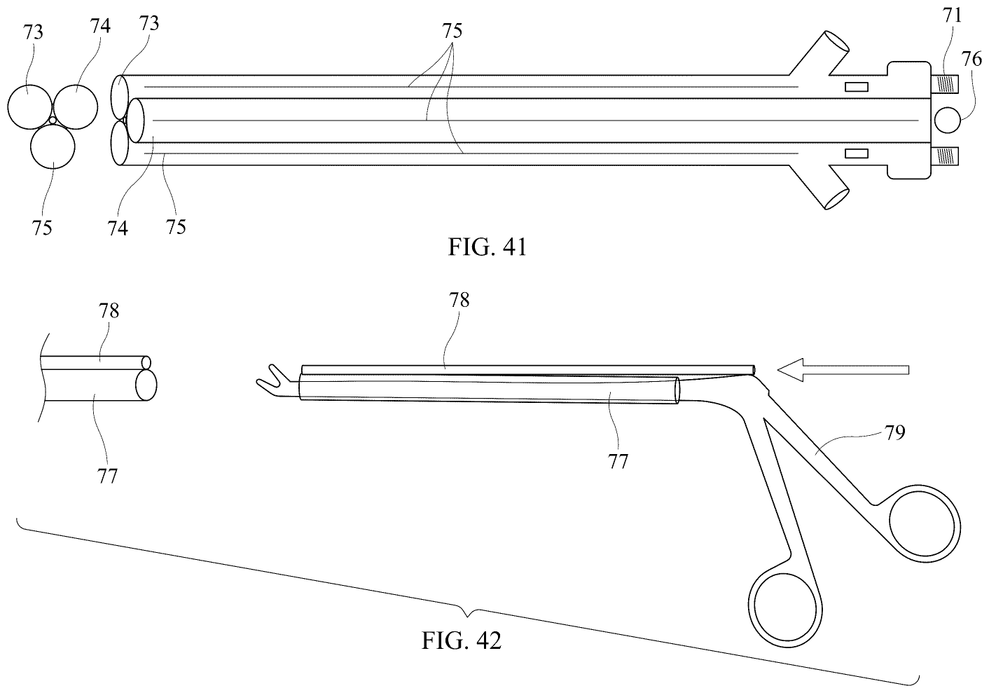

FIG. 41 shows another non-limiting example embodiment of the present system, in a tri-tube form, having three interconnected tubes 73, 74 and 75. In an example embodiment, some or all tubes 73, 74 and 75 may have built in metal Xray markers 75, which may also increase strength of a device of the present application. These embodiments may also be configured to include one or more irrigation and/or working instrument channels, at 71 and/or 76.

FIG. 42 shows an embodiment in which one tube 77 of a present FASTER device/system slides onto an instrument 79 and an interconnected tube 78 is available for insertion of a e.g., a scope.

According to example embodiments, the present systems and devices will have sterile and disposable packaging.

The present application also includes kits that include one or more of the FASTER devices of the present application, and optionally a further component related to use of the FASTER device. By way of non-limiting example, kits may optionally include one or more of the following: instructions for use, a storage container, a guidewire, a visualization tool, such as an endoscopic camera, and/or one or more working instruments, such as, but not limited to, a cautery device, motorized burr, shavers, and biters/pituitary instruments can be delivered accurately through the tissue.

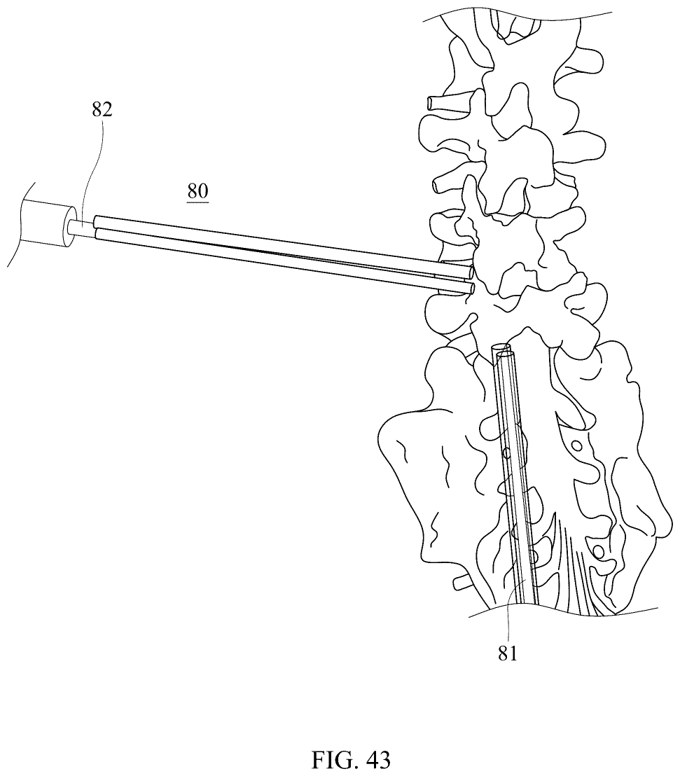

Also provided herein are methods of performing minimally invasive surgery, which include using the present FASTER devices and systems. By way of non-limiting example, FIG. 43 depicts how an example bi-portal dual cannula device 80 of the present application can be used when working in the neuroforamen for the transforaminal approach. In this example, a uniportal tube 81 can be driven to the lateral recess; using a technique such as driving a spinal cord stimulator wire. Once the uniportal tube 81 is in the appropriate position in a patient/subject, the “chip on a stick” scope 82 can be inserted with the dual cannula device 80 and used to identify e.g., the traversing nerve of the patient/subject. Once identified lateral to the nerve, the stiff stainless instruments can be passed to the area, used as a retractor; blocker to protect the nerve while the burr or a Kerrison removes the lateral stenosis.

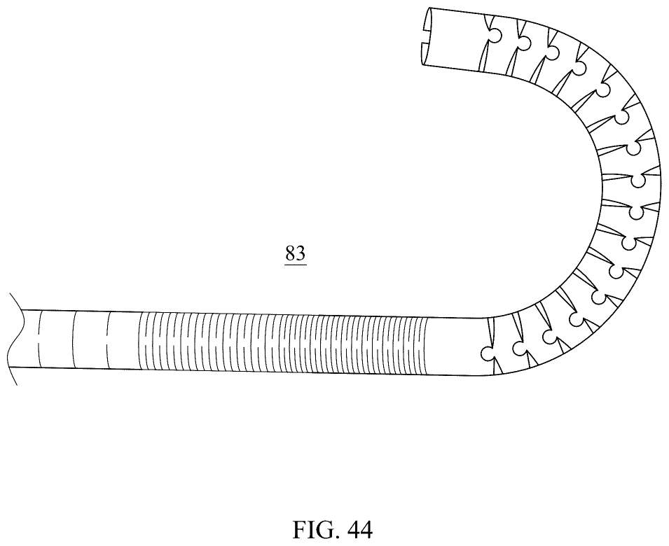

According to further example embodiments, an instrument 83 may be used with the present devices, systems and methods, and may optionally be included with the present kits, that can be placed within the tubes that can dial in varying flexibility, such as an adjustable flexible fingertip. See e.g., FIG. 44. The instrument may bend the tip of interconnected tubes in the present devices and systems to help navigate the spinal canal of a patient in the present methods. For example, a tip flexibility adjusting instrument may be used. When the one tube is flexed, the other interconnected tubes are connected, so they will follow suit. Or a flexible grasper can do the same.

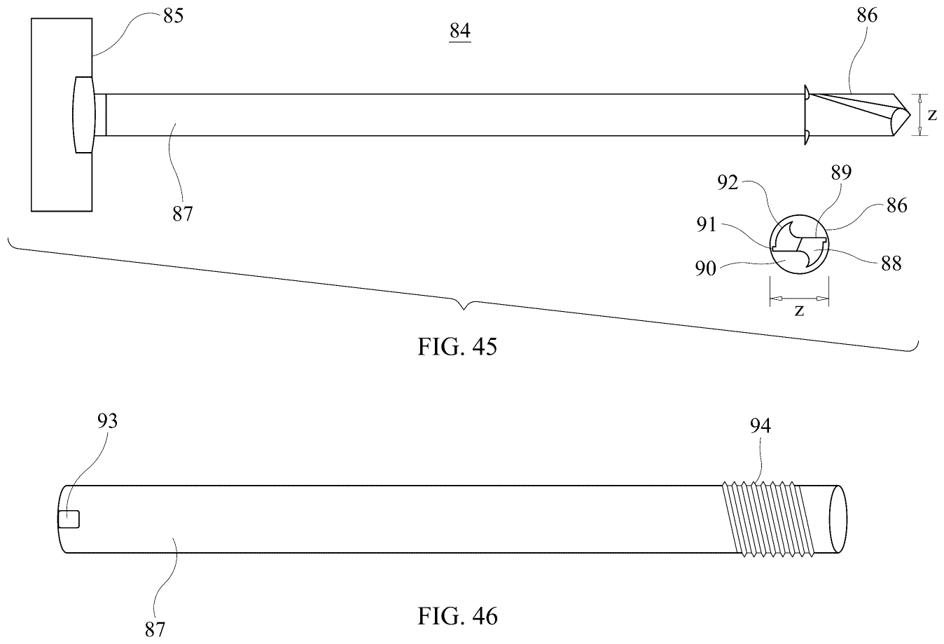

FIGS. 45-48 relate to additional FASTER instruments that may be used in accordance with example embodiments. In particular, FIGS. 45-48 show Standard Insertable Self Drilling Cannula on T-handle for Spine Instrument 84 passage.

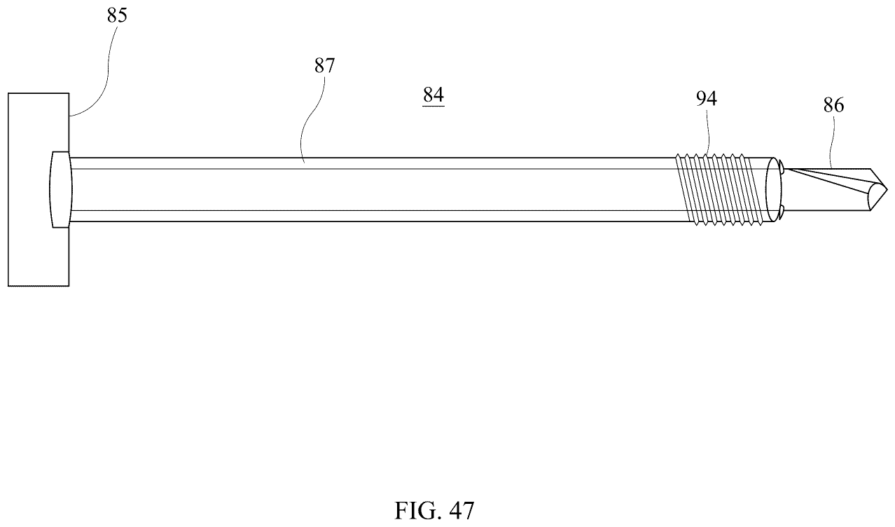

FIG. 45 depicts a T-handle 85 that can disconnect from a clip in metal solid stylet with a cutting flute 86 (Self Drilling), which flute 86 is also shown from a front view, to allow the cannula 84 to be driven with better accuracy through the pedicle or facet joint of a patient. Flute diameter z may be the same or approximately the same as the cannula 87 diameter, or the flute diameter z may be smaller than an inside diameter of the working channel cannula 87 so it may be inserted in the working channel cannula 87 as shown for example in FIG. 47. The flute 86 may include for example, a heel 88, lip 89, chisel edge or dead center 90, margin 91 and may have body clearance 92. Flutes having other configurations are also contemplated and included herein. Cross-section of the T-handle with clip in removable stylet component is not shown in image.

Although the present Figures and description show and refer to a T-handle, it is contemplated that the handle may be of a different shape, such as somewhat curved or angled or the handle ends may be of the same or different lengths.

FIG. 46 depicts the working channel cannula 87 that meets with the T-handle 85 to become one solid device when attached, which can be used to cannulate the facets and/or pedicle of a patient. These embodiments may be used for example in accessing the disc from the posterior approach or posterior lateral approach or far lateral approach. A lateral end of the working channel cannula 87 may include one or multiple locking tabs 93 for example, that the T-handle locks into the outer surface. For example, the locking tabs 93 may mate and lock the cannula 87 into the T-handle 85 or another handle. The opposite lateral side of the cannula 87 may include self-tapping threads, 94 which may be configured to screw into bone and lock the cannula in place in a patient.

In example embodiments, the cannula 84 may also have a small suction portal for inflow outflow to assist with bleeding.

FIG. 47 shows an assembled instrument according to example embodiments, locked in and ready for insertion through bone. Outside threading 94 of the cannula 87 will lock in bone and prevent bone bleeding from impeding endoscopic view. The Cannula 87 may be threaded and locked into bone; a metal cannula prevents cancellus bone bleeding from impeding trans-osseous visualization.

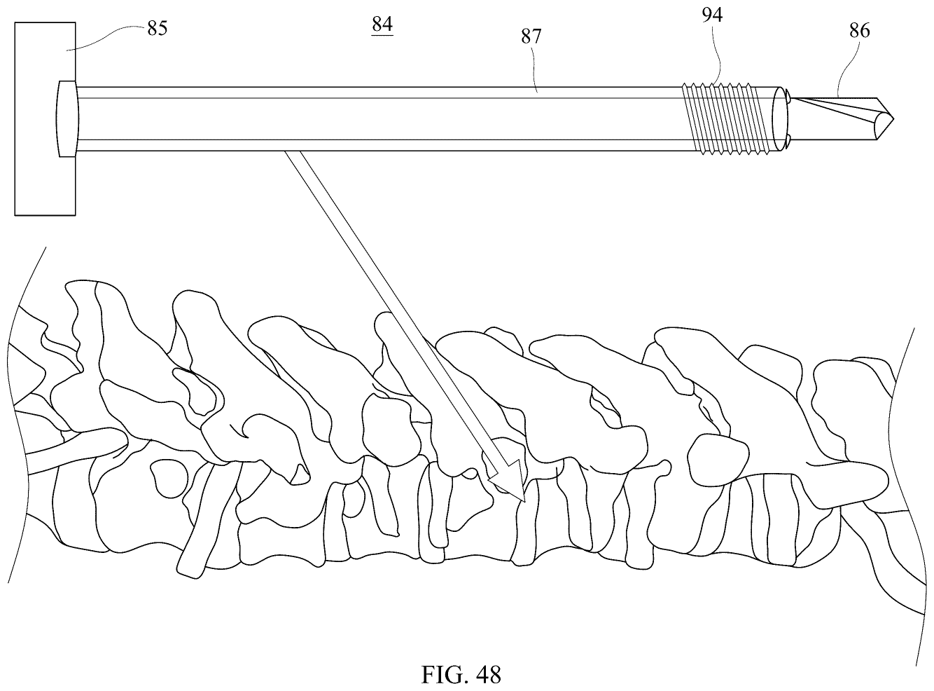

FIG. 48 shows embodiments of transpedicular and/or transforaminal to disc. The image shows a cannula 84 to be drilled through the pedicle to the posterior disc according to example embodiments. Embodiments include transpedicular access using radiologic imaging and/or navigation. Example embodiments allow access to the posterior disc. These embodiments may be used for example in a biportal endoscopic approach. Once the cannula 84 is in place, working instruments (Burr, Graspers, Kerrison's) can pass through the cannula and/or the endoscope for alternative visualizations for herniation removal and/or bone spur removal.

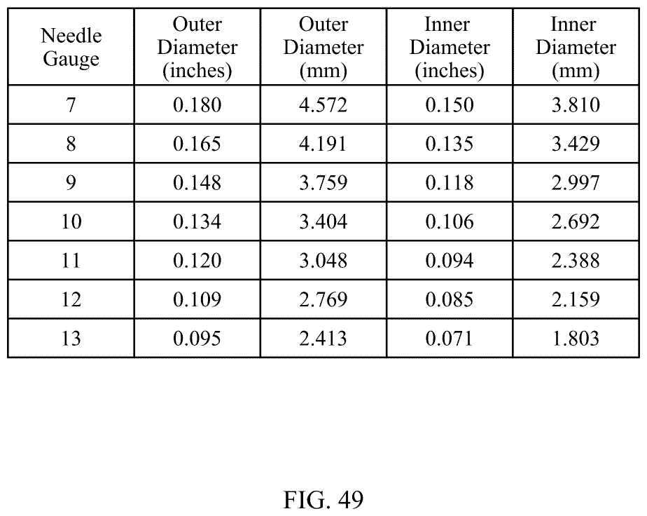

FIG. 49 is a table with diameters to show example outer and inner diameter of example cannulas for different cages in accordance with example embodiments. The diameter sizes of the present devices or tubes or cannulas that form part of the present devices, may be selected to allow passage of standard endoscopic instruments and cameras. Thoracic pedicles can range e.g. from 4-5-½ mm. Typically, a 9-12 gauge needle will be used. Instruments may be used and passed through this cannula with an example maximum outer diameter of 2.9 mm to limit the size of the overall cannula outer diameter to less than 4 mm or less than 4.5 mm.

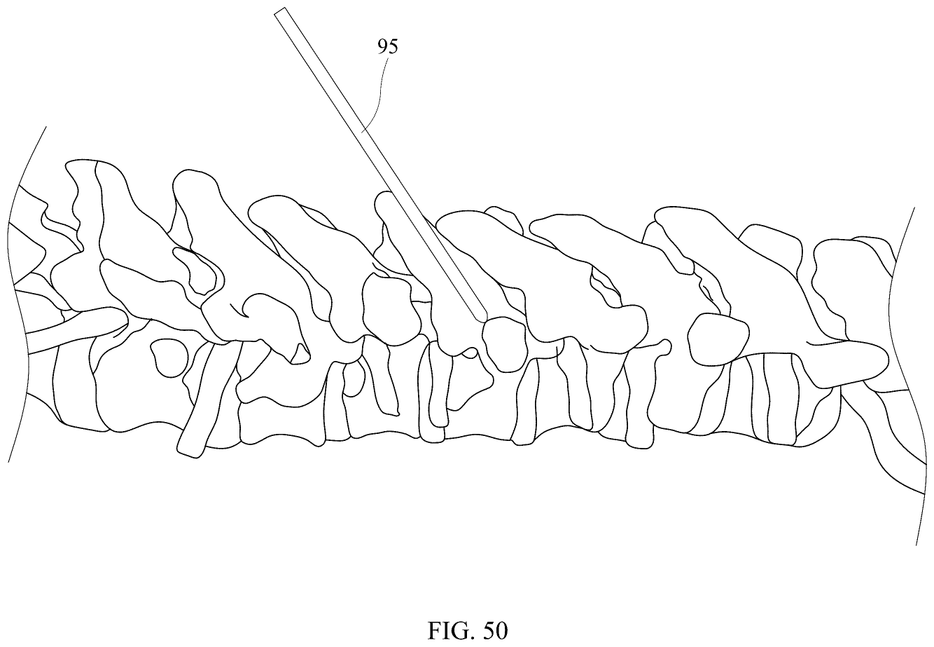

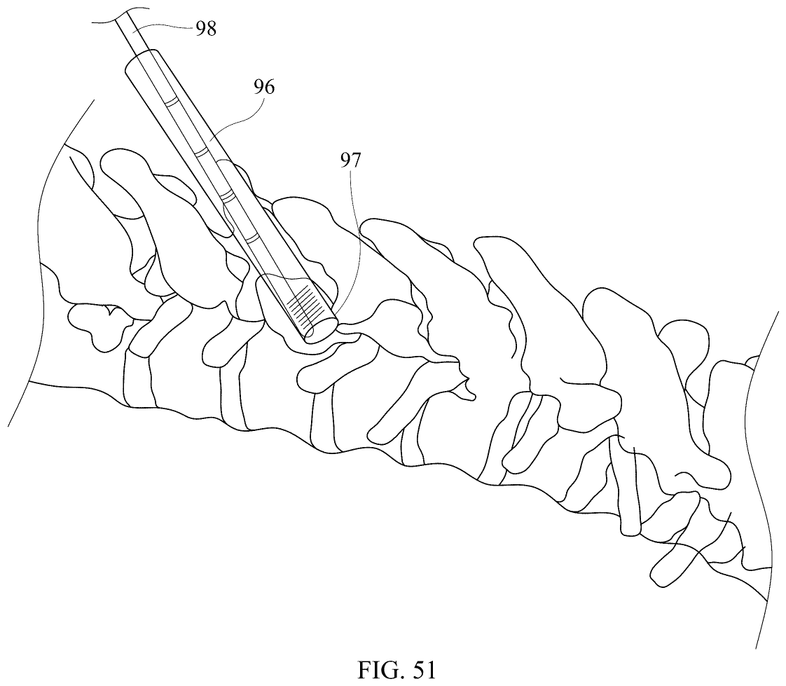

FIG. 50 shows an example of a portion 95 of a T-handle cannula drilling through sawbones and showing the trajectory of approach for radiographic guided transpedicular approach to remove disc pathology and/or bone spurs.

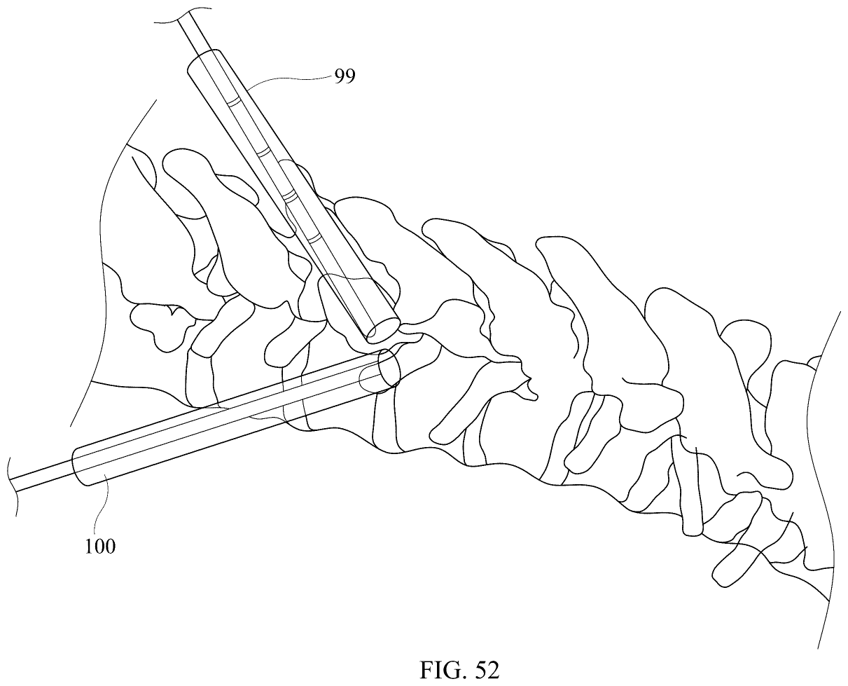

FIG. 51 shows an example of a cannula 96 locked in bone 97 (e.g. screwed into the bone) to allow working instruments 98 and/or a scope to be passed from skin down to the surgical site and posterior disc.

FIG. 52 shows example combined transpedicular and transforaminal approaches (bi-portal approach) to access posterior spinal structures. Using combined approaches limits the size of the cannulas that can be used, and allows smaller incisions to be made in alternating views of pathology. One can view the pathology from either of multiple tubes 99 or 100. A standard plastic polymer cannula may be used for example, as the transforaminal component 100. Embodiments of the present devices may be used e.g., as the transpedicular component 99. In example embodiments a fixed cannula system 100 may be an adjunct to a flexible system, 99. B-portal trans osseous approaches can be used within the spine by using the two types together.

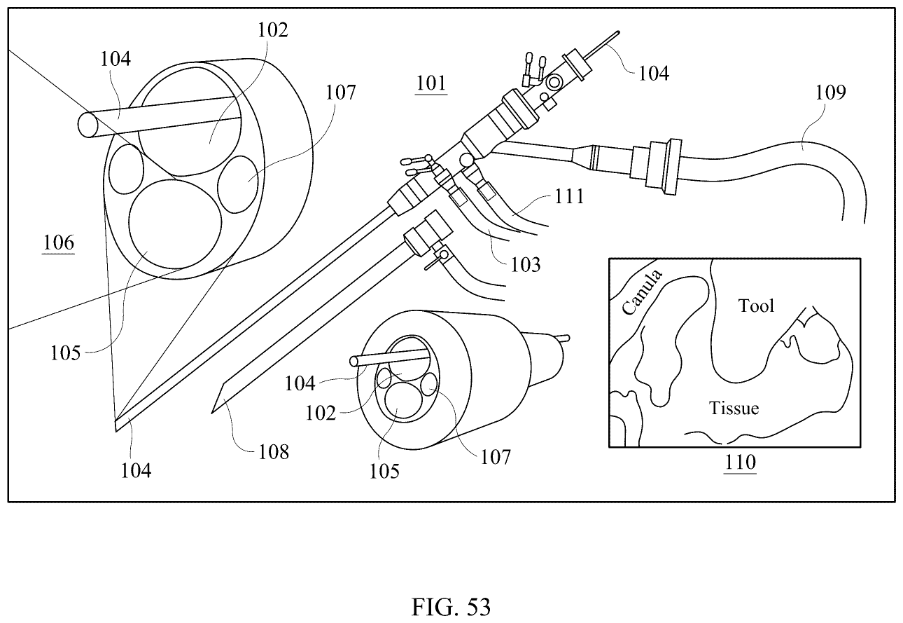

FIG. 53 shows an exploded view of a standard YESS (Yeung Endoscopic Spine Surgery System) endoscopic fixed working cannula arthroscope and its limitations compared to the novel FASTER features described herein. FIG. 53 shows a scope tip within a cannula 108, which includes a tool 104 within a working channel 102. A video cable 109 feeds into the discoscope and produces a video display 110. There is a video CCD pickup 105 having a video field 106. These systems also include an irrigation channel 107 in the scope tip and an irrigation port 103. There may further be a light cable 111. As can be sign in FIG. 53, this system is rigid.

The present application includes self-drilling cannula systems that include a cannula having a first lateral end and a second lateral end, a handle removably attachable to the first lateral end of the cannula, and a self-drilling cutting flute at the second lateral end of the cannula. According to example embodiments, the handle may be T-shaped. In non-limiting examples, the first lateral end of the cannula includes at least one locking tab configured to removably attach the handle to the first lateral end of the cannula. In example embodiments, an outer portion of the second lateral end of the cannula includes self-tapping threads configured to screw into bone and lock the cannula in place in a patient.

According to the present application, the patients or subjects are humans, but it is contemplated that the present devices may be used with other mammals.

In the examples of the present device, endoscopic spinal surgery or surgery on the extremities is discussed and exemplified, but it is contemplated that devices of the present system may be used or modified for use on other parts of the body, and for other minimally invasive or open surgeries.

While the present disclosure has been described in terms of exemplary aspects, those skilled in the art will recognize that the present disclosure can be practiced with modifications that are within the spirit and scope of the present application. The examples provided herein are merely illustrative and are not meant to be an exhaustive list of all possible designs, aspects, applications or modifications of the present disclosure. Accordingly, it is intended that such changes and modifications fall within the scope of the present disclosure

What is claimed is:

1. A flexible access supplementation tube for endoscopic exposure and retraction comprising:
at least a first flexible tube comprising a material suitable for insertion into a human, and
a second flexible tube comprising a material suitable for insertion into a human,
wherein the first and second flexible tubes are interconnected with each other, and
wherein at least one of the first flexible tube or the second flexible tube is a size suitable to accommodate an instrument therein.

2. The flexible access supplementation tube for endoscopic exposure and retraction of claim 1, wherein at least one of the first flexible tube or the second flexible tube comprises a braided expandible material.

3. The flexible access supplementation tube for endoscopic exposure and retraction of claim 1, wherein at least one of the first flexible tube or the second flexible tube comprises a braided expandible material.

4. The flexible access supplementation tube for endoscopic exposure and retraction of claim 1, further comprising a third flexible tube interconnected with the first and second flexible tubes.

5. The flexible access supplementation tube for endoscopic exposure and retraction of claim 1, further comprising a reinforcing metal strip in at least one of the first and second flexible tubes.

6. The flexible access supplementation tube for endoscopic exposure and retraction of claim 1, wherein at least one of the first and second flexible tubes comprises a braid composite tube having internal nitinol.

7. The flexible access supplementation tube for endoscopic exposure and retraction of claim 1, wherein at least one of the first and second flexible tubes further comprises an internal seal.

8. The flexible access supplementation tube for endoscopic exposure and retraction of claim 1, wherein at least one of the first and second flexible tubes further comprises an IV accessory tube.

9. The flexible access supplementation tube for endoscopic exposure and retraction of claim 1, further comprising a guide wire tunnel.

10. The flexible access supplementation tube for endoscopic exposure and retraction of claim 1, at least one of the first and second flexible tubes comprises an elastomer.

11. The flexible access supplementation tube for endoscopic exposure and retraction of claim 1, wherein at least one of the first and second flexible tubes comprises a reinforcing metal.

12. A kit comprising
at least one flexible access supplementation tube for endoscopic exposure and retraction according to claim 1, and
at least one additional component selected from the group consisting of instructions for use of the at least one flexible access supplementation tube for endoscopic exposure and retraction, a storage container, a guidewire, a visualization tool, and a working instrument.

13. The kit of claim 12, wherein the visualization tool comprises an endoscopic camera.

14. The kit of claim 12, wherein the working instrument comprises at least one working instrument selected from the group consisting of a cautery device, motorized burr, shavers, and a biters/pituitary instrument.

15. A method of treating a patient comprising
inserting a device into a patient, the device including at least a first flexible tube and a second flexible tube, wherein the first and second flexible tubes are interconnected with each other;
inserting a first instrument into the first flexible tube, to deliver the device to a predetermined location to a patient in need thereof.

16. The method of claim 15, wherein the first instrument has a scope at a lateral end thereof.

17. The method of claim 15, further comprising inserting a second instrument into the second flexible tube.

18. A self-drilling cannula system comprising
a cannula having a first lateral end and a second lateral end,
a handle removably attachable to the first lateral end of the cannula, and
a self-drilling cutting flute at the second lateral end of the cannula.

19. The self-drilling cannula system of claim 18, wherein the first lateral end of the cannula includes at least one locking tab configured to removably attach the handle to the first lateral end of the cannula.

20. The self-drilling cannula system of claim 18, wherein an outer portion of the second lateral end of the cannula includes self-tapping threads configured to screw the cannula into bone and lock the cannula in place in a patient.
